(19) United States
(12) Patent Application Publication
(10) Pub. No.: US 2007/0048372 A1
(43) Pub. Date: Mar. 1, 2007

(54) Method for treating non-inflammatory osteoarthritic pain

(75) Inventors: Bettina Beyreuther, Dusseldorf (DE); Thomas Stohr, Monheim (DE)

Correspondence Address: HARNESS, DICKEY, & PIERCE, P.L.C, 7700 BONHOMME, STE 400, ST. LOUIS, MO, 63105, US

(73) Assignee: SRZ Properties, Inc., Wilmington, DE (US)

(21) Appl. No.: 11/506,578

(22) Filed: Aug. 18, 2006

Related U.S. Application Data
(60) Provisional application No. 60/811,840, filed on Jun. 8, 2006.

(30) Foreign Application Priority Data
Aug. 18, 2005 (EP) EP 05 017 977.9

(51) Int. Cl. A61K 31/55; A61K 31/195; A61K 31/165; A61K 9/20

(52) U.S. Cl. 424/464; 514/616; 514/217; 514/389; 514/561

(57) ABSTRACT

A method for treating non-inflammatory osteoarthritic pain in a subject comprises administering to the subject a compound as defined herein, illustratively lacosamide, or a pharmaceutically acceptable salt thereof.

This application claims priority under 35 U.S.C. §119 of European Patent Application No. EP 05 017 977.9 filed on Aug. 18, 2005. This application also claims priority of U.S. provisional patent application Ser. No. 60/811,840, filed on Jun. 8, 2006. This application contains subject matter that is related to U.S. provisional patent application Ser. No. 60/811,859, filed on Jun. 8, 2006; to co-assigned U.S. application Ser. No. ______ titled “Method for treating non-inflammatory musculoskeletal pain”, filed concurrently herewith; to co-assigned U.S. application Ser. No. ______ titled “Therapeutic combination for painful medical conditions”, filed concurrently herewith; and to co-assigned U.S. application Ser. No. ______ titled “Combination therapy for pain in painful diabetic neuropathy”, filed concurrently herewith. The disclosure of each of the applications identified in this paragraph is incorporated herein by reference in its entirety.

### FIELD OF THE INVENTION

The present invention relates to therapeutic methods and combinations useful for treating non-inflammatory osteoarthritic pain.

### BACKGROUND OF THE INVENTION

Osteoarthritis is an acquired musculoskeletal disorder that is believed to be non-inflammatory in origin, occurring when the rate of cartilage degradation exceeds that of regeneration, resulting in cartilage erosion, subchondral bone thickening, and joint damage. As cartilage thins, its surface integrity can be lost, clefts can form, and the cartilage tends to be more easily eroded with joint motion. As new cartilage is formed, it tends to be more fibrous and less able to withstand mechanical stress. Over time, underlying bone can be exposed that is less capable of withstanding mechanical stress, resulting in microfractures. Localized osteonecrosis can occur beneath the bone surface, leading to cysts that can further weaken the bone's support of the cartilage.

As osteoarthritis progresses, it can eventually influence structures surrounding the joint. Local inflammation such as synovitis can occur, for example in response to inflammatory mediators released during the cartilage degradation process. The joint capsule tends to thicken, and movement of nutrients into and metabolic waste products out of the joint can be restricted. Eventually, periarticular muscle wasting can become evident as osteoarthritis progresses, and the joint is used less often or improperly. Pain of osteoarthritis is thought to be due not to cartilage degradation per se but to effects on surrounding structures including bone, since cartilage is aneural.

Subchondral bone, periosteum, synovium, ligaments, and the joint capsule are all richly innervated and contain nerve endings that could be source of nociceptive stimuli (Heppelmann (1997) J. Peripher. Nerv. Syst. 2(1):5-16; Mach et al. (2002) Neuroscience 113(1):155-166). In addition to peripheral pain sensitization, central pain sensitization can occur in osteoarthritis (Schaible et al. (2002) Ann. NY Acad. Sci. 966:343-354).

According to the Centers for Disease Control and Prevention (CDC), osteoarthritis is the most common form of arthritic disease, affecting 21 million Americans. See http://www.cdc.gov/arthritis/data_statistics/arthritis_related_statistics.htm#2.

By 2020, it is estimated that 60 million Americans will suffer from arthritis. Arthritis is the leading cause of physical disability (defined broadly as needing assistance in walking or climbing stairs) and of restricted daily activity in more than 7 million Americans, and this number is expected to grow to more than 11.6 million by 2020. See http://www.arthritis.org/resources/ActionPlanInterior.pdf.

It is very costly to treat arthritis and its complications. In 1997, the total cost of arthritis and other rheumatic conditions in the United States was $86 billion. The direct medical costs of arthritis and other rheumatic conditions in 1997 were $51.1 billion. The indirect costs (due to lost wages) of arthritis and other rheumatic conditions in 1997 were $35.1 billion. See http://www.cdc.gov/mmwr/preview/mmwrhtml/mm5318a3.htm.

The prevalence of osteoarthritis increases with age, and age is the largest risk factor. A survey reported by Brandt (2001) Principles of Internal Medicine, 15th ed. (Braunwald et al., eds.), New York: McGraw-Hill, pp. 1987-1994, found that only 2% of women less than 45 years old had radiographic evidence of osteoarthritis. In women aged 45 to 64 years, however, the prevalence was 30%, and for those 65 years or older it was 68%. Other risk factors include excess body weight, genetics, estrogen deficiency, repetitive joint use, and trauma.

A typical patient with osteoarthritis is middle-aged or elderly and complains of pain in the knee, hip, hand or spine. The distal and proximal interphalangeal joints of the hands are the most common sites of osteoarthritis but also the least likely to exhibit symptoms. The hip and knee are the second and third most common joints seen on X-ray to be affected, with knee pain being more likely to exhibit symptoms.

Pain is the paramount symptom of osteoarthritis. Osteoarthritic pain can have one or both of an inflammatory and a non-inflammatory component. Anti-inflammatory agents such as NSAIDs (non-steroidal anti-inflammatory drugs) and cyclooxygenase-2 inhibitors can be useful in treating or managing the inflammatory component, while opioid and other analgesics can be useful in treating or managing the non-inflammatory component. However, such drug therapies are not always effective and have side-effects that may not be well tolerated in all patients.

Non-inflammatory pain is often characterized by absence of swelling or warmth, absence of inflammatory or systemic features, and minimal or no morning stiffness.

Non-inflammatory osteoarthritic pain can contribute to a sedentary lifestyle, depression and sleep problems, particularly in the elderly. The pain is often characterized as a deep, aching sensation that intensifies with motion. It is usually intermittent and often mild, but can become persistent and severe. Crepitus is usually noted in the affected joints.

Certain peptides are known to exhibit central nervous system (CNS) activity and are useful in the treatment of epilepsy and other CNS disorders. Such peptides are described, for example, in U.S. Pat. No. 5,378,729.

Related peptides are disclosed in U.S. Pat. No. 5,773,475 as useful for treating CNS disorders.

International Patent Publication No. WO 02/074784, incorporated herein by reference in its entirety, relates to use of such peptide compounds having antinociceptive properties, for treatment of different types and symptoms of acute and chronic pain, especially non-neuropathic inflammatory pain, e.g., rheumatoid arthritic pain or secondary inflammatory osteoarthritic pain.

International Patent Publication No. WO 02/074297 relates to treatment of allodynia related to peripheral neuropathic pain, using a compound of formula 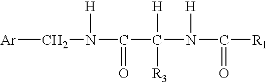
where Ar is a phenyl group that is unsubstituted or substituted with at least one halo substituent; R₃ is C₁₋₃ alkoxy; and R₁ is methyl.

Lacosamide (also called SPM 927 or harkoseride) is a compound of the above formula that has a mode of action which is not fully understood (Bialer et al. (2002) Epilepsy Res. 51:31-71). The mode of action of lacosamide and other peptide compounds disclosed in the above-referenced patents and publications differs from that of common antiepileptic drugs. Ion channels are not affected by these compounds in a manner comparable to other known antiepileptic drugs. For example, gamma-aminobutyric acid (GABA) induced currents are potentiated, but no direct interaction with any known GABA receptor subtype has been observed. Glutamate induced currents are attenuated but the compounds do not directly interact with any known glutamate receptor subtype.

A need remains for improved therapies that can treat non-inflammatory osteoarthritic pain.

### SUMMARY OF THE INVENTION

There is now provided a method for treating non-inflammatory osteoarthritic pain in a subject, comprising administering to the subject a compound of Formula (I) 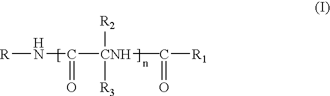
wherein:
R is hydrogen, lower alkyl, lower alkenyl, lower alkynyl, aryl, aryl lower alkyl, heterocyclic, heterocyclic lower alkyl, lower alkyl heterocyclic, lower cycloalkyl or lower cycloalkyl lower alkyl, and R is unsubstituted or is substituted with at least one electron withdrawing group, and/or at least one electron donating group;
R₁ is hydrogen or lower alkyl, lower alkenyl, lower alkynyl, aryl lower alkyl, aryl, heterocyclic lower alkyl, lower alkyl heterocyclic, heterocyclic, lower cycloalkyl, or lower cycloalkyl lower alkyl, and may be unsubstituted or substituted with at least one electron-withdrawing group and/or at least one electron-donating group;
R₂ and R₃ are independently hydrogen, lower alkyl, lower alkenyl, lower alkynyl, aryl lower alkyl, aryl, halo, heterocyclic, heterocyclic lower alkyl, lower alkyl heterocyclic, lower cycloalkyl, lower cycloalkyl lower alkyl, or Z-Y, wherein R₂ and R₃ are each independently unsubstituted or substituted with at least one electron-withdrawing group and/or at least one electron-donating group;
Z is O, S, S(O)ₐ, NR₄, NR₁₆, PR₄ or a chemical bond;
Y is hydrogen, lower alkyl, aryl, aryl lower alkyl, lower alkenyl, lower alkynyl, halo, heterocyclic, heterocyclic lower alkyl, or lower alkyl heterocyclic, and may be unsubstituted or substituted with at least one electron-withdrawing group and/or at least one electron-donating group, provided that when Y is halo, Z is a chemical bond, or
Z-Y taken together is NR₄NR₅R₇, NR₄OR₅, ONR₄R₇, OPR₄R₅, PR₄OR₅, SNR₄R₇, NR₄SR₇, SPR₄R₅, PR₄SR₇, NR₄PR₅R₆, PR4NR5R7, N⁺R₅R₆R₇, 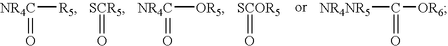
R′₆ is hydrogen, lower alkyl, lower alkenyl, or lower alkynyl, and may be unsubstituted or substituted with at least one electron-withdrawing group or/and at least one electron-donating group;
R₄, R₅ and R₆ are independently hydrogen, lower alkyl, aryl, aryl lower alkyl, lower alkenyl, or lower alkynyl, and are each independently unsubstituted or substituted with at least one electron-withdrawing group or/and at least one electron-donating group;
R₇ is R₆, COOR₈, or COR₈, and may be unsubstituted or substituted with at least one electron-withdrawing group or/and at least one electron-donating group;
R₈ is hydrogen, lower alkyl, or aryl lower alkyl, and may be unsubstituted or substituted with at least one electron-withdrawing group or/and at least one electron-donating group;
n is 1-4; and
a is 1-3;
   or a pharmaceutically acceptable salt thereof.

An illustrative compound of Formula (I) is lacosamide, (R)-2-acetamido-N-benzyl-3-methoxypropionamide.

Other embodiments, including particular aspects of the embodiments summarized above, will be evident from the detailed description that follows.

### BRIEF DESCRIPTION OF THE DRAWINGS

In FIGS. 1-4, “SPM 927” refers to lacosamide.

FIG. 1 is a graphical representation of results of the study of Example 1, showing effect of lacosamide at 3, 10 and 30 mg/kg on muscle pressure hyperalgesia induced by TNF.

FIG. 2 is a graphical representation of results of the study of Example 1, showing maximal possible effect (MPE) of lacosamide at 3, 10 and 30 mg/kg, in comparison to pregabalin, gabapentin and metamizol (dipyrone), on muscle pressure hyperalgesia induced by TNF.

FIG. 3 is a graphical representation of results of the study of Example 1, effect of lacosamide at 3, 10 and 30 mg/kg on biceps muscle grip strength after TNF-induced muscle pain.

FIG. 4 is a graphical representation of results of the study of Example 1, showing maximal possible effect (MPE) of lacosamide at 3, 10 and 30 mg/kg, in comparison to pregabalin, gabapentin and metamizol (dipyrone), on biceps muscle grip strength after TNF-induced muscle pain.

FIGS. 5A-C are graphical representations of results of the study of Example 2, showing effects of lacosamide and morphine on monosodium iodoacetate-induced tactile allodynia at days 3, 7 and 14 of the study respectively.

FIGS. 6A-C are graphical representations of results of the study of Example 2, showing effect of diclofenac on monosodium iodoacetate-induced tactile allodynia at days 3, 7 and 14 of the study respectively.

FIGS. 7A-C are graphical representations of results of the study of Example 2, showing effects of lacosamide and morphine on monosodium iodoacetate-induced mechanical hyperalgesia at days 3, 7 and 14 of the study respectively.

FIGS. 8A-C are graphical representations of results of the study of Example 2, showing effect of diclofenac on monosodium iodoacetate-induced mechanical hyperalgesia at days 3, 7 and 14 of the study respectively.

### DETAILED DESCRIPTION

Stöhr et al. (2006) Eur. J. Pain 10(3):241-249, incorporated herein by reference in its entirety but not admitted to be prior art to the present invention, describes results of studies of lacosamide in animal models for inflammatory pain.

Methods and therapeutic combinations provided herein are useful for treating non-inflammatory osteoarthritic pain. Non-inflammatory osteoarthritic pain is a specific type of non-inflammatory musculoskeletal pain which typically arises from effects of osteoarthritis-related morphological alterations, such as cartilage degradation, structural bone changes, and vascularization of areas of osteoarthritic bone remodeling. It is distinguished herein from inflammatory osteoarthritic pain, which typically occurs from synovial inflammation following pathological processes in cartilage and bone involving tissue damage and macrophage infiltration (resulting in edema) associated with a classical immune system response.

Non-inflammatory osteoarthritic pain may also be associated with and/or caused by a pathological condition. Such conditions include, for example without limitation, regional pain syndrome such as back or neck pain, rheumatoid arthritis, osteoarthritis, gout, ankylosing spondylitis, lupus erythematosus, fibromyalgia, fibrositis, fibromyositis, myofascial pain syndrome, autoimmune disorders, polymyalgia rheumatica, polymyositis, dermatomyositis, muscular abscess, trichinosis, Lyme disease, malaria, Rocky Mountain spotted fever, polio, trauma, joint damage and joint damage by trauma.

Unless the context demands otherwise, the term “treat,” “treating” or “treatment” herein includes preventive or prophylactic use of an agent, for example a compound of Formula (I), in a subject at risk of, or having a prognosis including, non-inflammatory osteoarthritic pain, as well as use of such an agent in a subject already experiencing non-inflammatory osteoarthritic pain, as a therapy to alleviate, relieve, reduce intensity of or eliminate such pain or an underlying cause thereof.

The term “subject” refers to a warm-blooded animal, generally a mammal such as, for example, a cat, dog, horse, cow, pig, mouse, rat or primate, including a human. In one embodiment the subject is a human, for example a patient having clinically diagnosed osteoarthritis.

The compound administered according to the present method is a compound of Formula (I) as set forth above, or a pharmaceutically acceptable salt thereof. Terms used in the description of Formula (I) and elsewhere in the present specification unless otherwise indicated, are defined as follows.

The term “alkyl,” alone or in combination with another term(s), means a straight- or branched-chain saturated hydrocarbyl substituent typically containing from 1 to about 20 carbon atoms, more typically from 1 to about 8 carbon atoms, and even more typically from 1 to about 6 carbon atoms.

The term “lower alkyl” refers to an alkyl substituent containing from 1 to 6 carbon atoms, especially 1 to 3 carbon atoms, that may be straight-chain or branched. Examples include methyl, ethyl, propyl, isopropyl, butyl, isobutyl, tertiary butyl, pentyl, hexyl, and the like, and isomers thereof.

The term “alkenyl,” alone or in combination with another term(s), means a straight- or branched-chain hydrocarbyl substituent containing one or more double bonds and typically from 2 to about 20 carbon atoms, more typically from 2 to about 8 carbon atoms, and even more typically from 2 to about 6 carbon atoms. Alkenyl groups, where asymmetric, can have cis or trans configuration.

The term “lower alkenyl” refers to an alkenyl substituent containing from 2 to 6 carbon atoms that may be straight-chained or branched and in the Z or E form. Examples include vinyl, propenyl, 1-butenyl, isobutenyl, 2-butenyl, 1-pentenyl, (Z)-2-pentenyl, (E)-2-pentenyl, (Z)-4-methyl-2-pentenyl, (E)-4-methyl-2-pentenyl, pentadienyl, e.g., 1, 3 or 2,4-pentadienyl, and the like.

The term “alkynyl,” alone or in combination with another term(s), means a straight- or branched-chain hydrocarbyl substituent containing one or more triple bonds and typically from 2 to about 20 carbon atoms, more typically from 2 to about 8 carbon atoms, and even more typically from 2 to about 6 carbon atoms.

The term “lower alkynyl” refers to an alkynyl substituent containing 2 to 6 carbon atoms that may be straight-chained or branched. It includes such groups as ethynyl, propynyl, 1-butynyl, 2-butynyl, 1-pentynyl, 2-pentynyl, 3-methyl-1-pentynyl, 3-pentynyl, 1-hexynyl, 2-hexynyl, 3-hexynyl and the like.

The term “cycloalkyl,” alone or in combination with another term(s), means a completely or partially saturated alicyclic hydrocarbyl group containing from 3 to about 18 ring carbon atoms. Cycloalkyl groups may be monocyclic or polycyclic. Examples include cyclopropyl, cyclobutyl, cyclopentyl, cyclohexyl, cycloheptyl, cyclooctyl, cyclodecyl, cyclohexenyl, cyclopentenyl, cyclooctenyl, cycloheptenyl, decalinyl, hydroindanyl, indanyl, fenchyl, pinenyl, adamantyl, and the like. Cycloalkyl includes the cis or trans forms. Cycloalkyl groups may be unsubstituted or mono- or polysubstituted with electron withdrawing or/and electron donating groups as described below. Furthermore, the substituents may either be in endo- or exo-positions in bridged bicyclic systems. “Lower cycloalkyl” groups have 3 to 6 carbon atoms.

The term “alkoxy,” alone or in combination with another term(s), means an alkylether, i.e., —O-alkyl, substituent.

The term “lower alkoxy” refers to an alkoxy substituent containing from 1 to 6 carbon atoms, especially 1 to 3 carbon atoms, that may be straight-chain or branched. Examples include methoxy, ethoxy, propoxy, butoxy, isobutoxy, tert-butoxy, pentoxy, hexoxy and the like.

The term “aryl,” alone or in combination with another term(s), means an aromatic group which contains from about 6 to about 18 ring carbon atoms, and includes polynuclear aromatics. Aryl groups may be monocyclic or polycyclic, and optionally fused. A polynuclear aromatic group as used herein encompasses bicyclic and tricyclic fused aromatic ring systems containing from about 10 to about 18 ring carbon atoms. Aryl groups include phenyl, polynuclear aromatic groups (e.g., naphthyl, anthracenyl, phenanthrenyl, azulenyl and the like), and groups such as ferrocenyl. Aryl groups may be unsubstituted or mono- or polysubstituted with electron-withdrawing and/or electron-donating groups as described below.

“Aryl lower alkyl” groups include, for example, benzyl, phenylethyl, phenylpropyl, phenylisopropyl, phenylbutyl, diphenylmethyl, 1,1-diphenylethyl, 1,2-diphenylethyl, and the like.

The term “monosubstituted amino,” alone or in combination with another term(s), means an amino substituent wherein one of the hydrogen radicals is replaced by a non-hydrogen substituent. The term “disubstituted amino,” alone or in combination with another term(s), means an amino substituent wherein both of the hydrogen atoms are replaced by non-hydrogen substituents, which may be identical or different.

The term “halo” or “halogen” includes fluoro, chloro, bromo, and iodo.

The term “carbalkoxy” refers to —CO—O-alkyl, wherein alkyl may be lower alkyl as defined above.

The prefix “halo” indicates that the substituent to which the prefix is attached is substituted with one or more independently selected halogen radicals. For example, haloalkyl means an alkyl substituent wherein at least one hydrogen radical is replaced with a halogen radical. Examples of haloalkyl substituents include chloromethyl, 1-bromoethyl, fluoromethyl, difluoromethyl, trifluoromethyl, 1,1,1-trifluoroethyl, and the like. Illustrating further, “haloalkoxy” means an alkoxy substituent wherein at least one hydrogen radical is replaced by a halogen radical. Examples of haloalkoxy substituents include chloromethoxy, 1-bromoethoxy, fluoromethoxy, difluoromethoxy, trifluoromethoxy (also known as “perfluoromethyloxy”), 1,1,1,-trifluoroethoxy, and the like. It should be recognized that if a substituent is substituted with more than one halogen radical, those halogen radicals may be identical or different, unless otherwise stated.

The term “acyl” includes alkanoyl containing from 1 to about 20 carbon atoms, preferably 1 to 6 carbon atoms, and may be straight-chain or branched. Acyl groups include, for example, formyl, acetyl, propionyl, butyryl, isobutyryl, tertiary butyryl, pentanoyl and isomers thereof, and hexanoyl and isomers thereof.

The terms “electron-withdrawing” and “electron-donating” refer to the ability of a substituent to withdraw or donate electrons, respectively, relative to that of hydrogen if a hydrogen atom occupied the same position in the molecule. These terms are well understood by one skilled in the art and are discussed, for example, in March (1985), Advanced Organic Chemistry, New York: John Wiley & Sons, at pp. 16-18, the disclosure of which is incorporated herein by reference. Electron-withdrawing groups include halo (including fluoro, chloro, bromo, and iodo), nitro, carboxy, lower alkenyl, lower alkynyl, formyl, carboxyamido, aryl, quaternary ammonium, haloalkyl (such as trifluoromethyl), aryl lower alkanoyl, carbalkoxy, and the like. Electron-donating groups include hydroxy, lower alkoxy (including methoxy, ethoxy, and the like), lower alkyl (including methyl, ethyl, and the like), amino, lower alkylamino, di(lower alkyl)amino, aryloxy (such as phenoxy), mercapto, lower alkylthio, lower alkylmercapto, disulfide (lower alkyldithio), and the like. One of ordinary skill in the art will appreciate that some of the aforesaid substituents may be considered to be electron-donating or electron-withdrawing under different chemical conditions. Moreover, the present invention contemplates any combination of substituents selected from the above-identified groups.

The term “heterocyclic” means a ring substituent that contains one or more sulfur, nitrogen and/or oxygen ring atoms. Heterocyclic groups include heteroaromatic groups and saturated and partially saturated heterocyclic groups. Heterocyclic groups may be monocyclic, bicyclic, tricyclic or polycyclic and can be fused rings. They typically contain up to 18 ring atoms, including up to 17 ring carbon atoms, and can contain in total up to about 25 carbon atoms, but preferably are 5- to 6-membered rings. Heterocyclic groups also include the so-called benzoheterocyclics. Representative heterocyclic groups include furyl, thienyl, pyrazolyl, pyrrolyl, methylpyrrolyl, imidazolyl, indolyl, thiazolyl, oxazolyl, isothiazolyl, isoxazolyl, piperidyl, pyrrolinyl, piperazinyl, quinolyl, triazolyl, tetrazolyl, isoquinolyl, benzofuryl, benzothienyl, morpholinyl, benzoxazolyl, tetrahydrofuryl, pyranyl, indazolyl, purinyl, indolinyl, pyrazolindinyl, imidazolinyl, imadazolindinyl, pyrrolidinyl, furazanyl, N-methylindolyl, methylfuryl, pyridazinyl, pyrimidinyl, pyrazinyl, pyridyl, epoxy, aziridino, oxetanyl, and azetidinyl groups, as well as N-oxides of nitrogen-containing heterocyclics, such as the N-oxides of pyridyl, pyrazinyl, and pyrimidinyl groups and the like. Heterocyclic groups may be unsubstituted or mono- or polysubstituted with electron-withdrawing and/or electron-donating groups.

In one embodiment, a heterocyclic group is selected from thienyl, furyl, pyrrolyl, benzofuryl, benzothienyl, indolyl, methylpyrrolyl, morpholinyl, pyridyl, pyrazinyl, imidazolyl, pyrimidinyl, and pyridazinyl, especially furyl, pyridyl, pyrazinyl, imidazolyl, pyrimidinyl, and pyridazinyl, more especially furyl and pyridyl.

In another embodiment, a heterocyclic group is selected from furyl, optionally substituted with at least one lower alkyl group (preferably one having 1-3 carbon atoms, for example methyl), pyrrolyl, imidazolyl, pyridyl, pyrazinyl, pyrimidinyl, oxazolyl and thiazolyl, especially furyl, pyridyl, pyrazinyl, pyrimidinyl, oxazolyl and thiazolyl, more especially furyl, pyridyl, pyrimidinyl and oxazolyl.

Illustratively, in the compound of Formula (I) n is 1, but di- (n=2), tri- (n=3) and tetrapeptides (n=4) are also contemplated to be useful herein.

R in the compound of Formula (I) is illustratively aryl lower alkyl, especially benzyl where the phenyl ring thereof is unsubstituted or substituted with one or more electron-donating groups and/or electron-withdrawing groups, such as halo (e.g., fluoro).

R₁ in the compound of Formula (I) is preferably hydrogen or lower alkyl, especially methyl.

Particularly suitable electron-withdrawing and/or electron-donating substituents are halo, nitro, alkanoyl, formyl, arylalkanoyl, aryloyl, carboxyl, carbalkoxy, carboxamido, cyano, sulfonyl, sulfoxide, heterocyclic, guanidine, quaternary ammonium, lower alkenyl, lower alkynyl, sulfonium salts, hydroxy, lower alkoxy, lower alkyl, amino, lower alkylamino, di(lower alkyl)amino, amino lower alkyl, mercapto, mercaptoalkyl, alkylthio, and alkyldithio. The term “sulfide” encompasses mercapto, mercapto alkyl and alkylthio, while the term disulfide encompasses alkylthio. Preferred electron-withdrawing and/or electron-donating groups are halo and lower alkoxy, especially fluoro and methoxy. These preferred substituents may be present in any one or more of the groups R, R₁, R₂, R₃, R₄, R₅, R₆, R′₆, R₇ or R₈ as defined herein.

Z-Y groups representative of R₂ and/or R₃ include hydroxy, alkoxy (such as methoxy and ethoxy), aryloxy (such as phenoxy), thioalkoxy (such as thiomethoxy and thioethoxy), thioaryloxy (such as thiophenoxy), amino, alkylamino (such as methylamino and ethylamino), arylamino (such as anilino), lower dialkylamino (such as dimethylamino), trialkylammonium salt, hydrazino, alkylhydrazino and arylhydrazino (such as N-methylhydrazino and N-phenylhydrazino), carbalkoxy hydrazino, aralkoxycarbonyl hydrazino, aryloxycarbonyl hydrazino, hydroxylamino (such as N-hydroxylamino (—NHOH)), lower alkoxyamino (NHOR₁₈ wherein R₁₈ is lower alkyl, e.g., methyl), N-lower alkylhydroxylamino (N(R₁₈)OH wherein R₁₈ is lower alkyl), N-lower alkyl-O-lower alkylhydroxylamino (N(R₁₈)OR₁₉ wherein R₁₈ and R₁₉ are independently lower alkyl), and o-hydroxylamino (—O—NH₂)), alkylamido (such as acetamido), trifluoroacetamido, and heterocyclylamino (such as pyrazoylamino).

Preferred heterocyclic groups representative of R₂ and/or R₃ are monocyclic 5- or 6-membered heterocyclic moieties of the formula 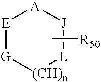
including unsaturated, partially and fully saturated forms thereof, wherein n is 0 or 1; R₅₀ is hydrogen or an electron-withdrawing or electron-donating group; A, E, L, J and G are independently CH, or a heteroatom selected from the group consisting of N, O and S; but when n is 0, G is CH, or a heteroatom selected from the group consisting of N, O and S; with the proviso that at most two of A, E, L, J and G are heteroatoms.

If n is 0, the above monocyclic heterocyclic ring is 5-membered, while if n is 1, the ring is 6-membered.

If the ring depicted hereinabove contains a nitrogen ring atom, then the N-oxide forms are also contemplated to be within the scope of the invention.

When R₂ or R₃ comprises a heterocyclic group of the above formula, it may be bonded to the main chain by a ring carbon atom. When n is 0, R₂ or R₃ may additionally be bonded to the main chain by a nitrogen ring atom.

Other preferred moieties of R₂ and R₃ are hydrogen, aryl (e.g., phenyl), arylalkyl (e.g., benzyl), and alkyl. Such moieties can be unsubstituted or mono- or polysubstituted with electron-withdrawing and/or electron-donating groups. In various embodiments, R₂ and R₃ are independently hydrogen; lower alkyl, either unsubstituted or substituted with one or more electron-withdrawing and/or electron-donating groups such as lower alkoxy (e.g., methoxy, ethoxy, and the like); N-hydroxylamino; N-lower alkylhydroxyamino; N-lower alkyl-O-lower alkyl; or alkylhydroxylamino.

In some embodiments, one of R₂ and R₃ is hydrogen.

In one embodiment n in Formula (I) is 1 and one of R₂ and R₃ is hydrogen. Illustratively in this embodiment, R₂ is hydrogen and R₃ is lower alkyl or Z-Y where Z is O, NR₄ or PR₄, and Y is hydrogen or lower alkyl; or Z-Y is NR₄NR₅R₇, NR₄OR₅, ONR₄R₇,

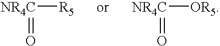

In another embodiment, n is 1, R₂ is hydrogen, and R₃ is lower alkyl which is unsubstituted or substituted with an electron-withdrawing or electron-donating group, NR₄OR₅, or ONR₄R₇.

In yet another embodiment,
n is 1;
R is aryl lower alkyl, which aryl group is unsubstituted or substituted with an electron-withdrawing group, for example aryl can be phenyl, which is unsubstituted or substituted with halo;
R₁ is lower alkyl;
R₂ is hydrogen; and
R₃ is lower alkyl which is unsubstituted or substituted with hydroxy, lower alkoxy, NR₄OR₅ or ONR₄R₇, wherein R₄, R₅ and R₇ are independently hydrogen or lower alkyl.

In yet another embodiment, R₂ is hydrogen and R₃ is hydrogen, an alkyl group which is unsubstituted or substituted with at least one electron-withdrawing or electron-donating group or Z-Y. In this embodiment, R₃ is illustratively hydrogen, an alkyl group such as methyl, which is unsubstituted or substituted with an electron-donating group such as lower alkoxy, more especially methoxy or ethoxy, or with NR₄OR₅ or ONR₄R₇, wherein R₄, R₅ and R₇ are independently hydrogen or lower alkyl.

In yet another embodiment, R₂ and R₃ are independently hydrogen, lower alkyl, or Z-Y; Z is O, NR₄ or PR₄; Y is hydrogen or lower alkyl; or Z-Y is NR₄NR₅R₇, NR₄OR₅, ONR₄R₇,

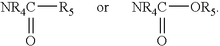

It is preferred that R is aryl lower alkyl. The most preferred aryl for R is phenyl. The most preferred R group is benzyl. The aryl group is unsubstituted or substituted with an electron-withdrawing or electron-donating group. If the aryl ring in R is substituted, it is most preferred that it is substituted with an electron-withdrawing group, The most preferred electron-withdrawing group for R is halo, especially fluoro.

The preferred R₁ is lower alkyl, especially methyl.

In one embodiment R is aryl lower alkyl, e.g., benzyl, and R₁ is lower alkyl, e.g., methyl.

Further preferred compounds are compounds of Formula (I) wherein
n is 1;
R is aryl or aryl lower alkyl, such as benzyl, wherein the aryl group is unsubstituted or substituted with an electron-withdrawing or electron-donating group;
R₁ is lower alkyl;
R₂ is hydrogen; and
R₃ is hydrogen, a lower alkyl group, especially methyl which is substituted with an electron-withdrawing or electron-donating group, or Z-Y.
   In this embodiment, it is more preferred that R₃ is hydrogen, a lower alkyl group, especially methyl, which may be substituted with an electron-donating group such as lower alkoxy (e.g., methoxy, ethoxy or the like), NR₄OR₅ or ONR₄R₇ wherein these groups are as defined hereinabove.

In one aspect, the compound is represented by Formula (II) 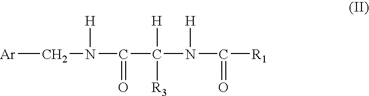
or a pharmaceutically acceptable salt thereof, wherein
Ar is aryl, especially phenyl, which is unsubstituted or substituted with at least one halo;
R₁ is lower alkyl, especially C₁₋₃ alkyl, for example methyl; and
R₃ is hydrogen or lower alkyl, which is unsubstituted or substituted with at least one electron-withdrawing or electron-donating group or Z-Y; for example R₃ is —CH₂-Q, wherein Q is lower alkoxy, especially C₁₋₃ alkoxy, for example methoxy.

In another aspect, the compound has formula (I) wherein
n is 1;
R is unsubstituted or substituted benzyl, in particular halo-substituted benzyl;
R₁ is lower alkyl, especially C₁₋₃ alkyl, for example methyl;
R₂ is hydrogen; and
R₃ is as broadly defined herein.

In yet another aspect, the compound is represented by Formula (III) 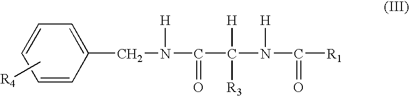
or a pharmaceutically acceptable salt thereof, wherein
R₄ is one or more substituents independently selected from the group consisting of hydrogen, halo, alkyl, alkenyl, alkynyl, nitro, carboxy, formyl, carboxyamido, aryl, quaternary ammonium, haloalkyl, aryl alkanoyl, hydroxy, alkoxy, amino, alkylamino, dialkylamino, aryloxy, mercapto, alkylthio, alkylmercapto, and disulfide;
R₃ is selected from the group consisting of hydrogen, alkyl, alkoxy, alkoxyalkyl, aryl, N-alkoxy-N-alkylamino, and N-alkoxyamino; and
R₁ is alkyl.

Alkyl, alkoxy, alkenyl and alkynyl groups in a compound of Formula (III) are lower alkyl, alkoxy, alkenyl and alkynyl groups having no more than 6, more typically no more than 3, carbon atoms.

In a particular aspect, R₄ substituents in a compound of Formula (III) are independently selected from hydrogen and halo, more particularly fluoro, substituents.

In a particular aspect, R₃ in a compound of Formula (III) is alkoxyalkyl, phenyl, N-alkoxy-N-alkylamino or N-alkoxyamino.

In a particular aspect, R₁ in a compound of Formula (III) is C₁₋₃ alkyl.

In a more particular aspect, no more than one R₄ substituent is fluoro and all others are hydrogen; R₃ is selected from the group consisting of methoxymethyl, phenyl, N-methoxy-N-methylamino and N-methoxyamino; and R₁ is methyl.

It is to be understood that combinations and permutations of R₁, R₂, R₃ and R groups and values of n, even if such combinations and permutations are not explicitly described herein, are contemplated to be within the scope of the present invention. Moreover, the present invention also encompasses methods that comprise administering a compound having one or more elements of each of the Markush groupings described for R₁, R₂, R₃ and R and the various combinations thereof. Thus, for example, the present invention contemplates that R₁ and R may independently be one or more of the substituents listed hereinabove in combination with any of the R₂ and R₃ substituents, independently with respect to each of the n 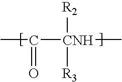
subunits of the compound of Formula (I).

Compounds useful herein may contain one or more asymmetric carbons and may exist in optically active forms. The configuration around each asymmetric carbon can be either the D or L configuration. Configuration around a chiral carbon atom can also be described as R or S in the Cahn-Prelog-Ingold system. All of the various configurations around each asymmetric carbon, including the various enantiomers and diastereomers as well as mixtures of enantiomers, diastereomers or both, including but not limited to racemic mixtures, are contemplated by the present invention.

More particularly, in a compound of Formula (I) where R₂ and R₃ are not identical, there exists asymmetry at the carbon atom to which the groups R₂ and R₃ are attached. As used herein, the term “configuration” generally refers to the configuration around the carbon atom to which R₂ and R₃ are attached, even though other chiral centers may be present in the molecule. Therefore, unless the context demands otherwise, when referring to a particular configuration such as D or L, it is to be understood to mean the D- or L-stereoisomer at the carbon atom to which R₂ and R₃ are attached. However, all possible enantiomers and diastereomers at other chiral centers, if any, present in the compound are encompassed herein.

The compounds useful herein can comprise the L- or D-stereoisomer as defined above, or any mixture thereof, including without limitation a racemic mixture. The D-stereoisomer is generally preferred. In lacosamide, the D-stereoisomer corresponds to the R-enantiomer according to R,S terminology.

In one embodiment the compound, for example lacosamide, is substantially enantiopure. As used herein, the term “substantially enantiopure” means having at least 88%, preferably at least 90%, more preferably at least 95%, 96%, 97%, 98% or 99% enantiomeric purity.

Illustrative compounds that can be used according to the present method include:
(R)-2-acetamido-N-benzyl-3-methoxypropionamide (lacosamide);
(R)-2-acetamido-N-benzyl-3-ethoxypropionamide;
O-methyl-N-acetyl-D-serine-m-fluorobenzylamide;
O-methyl-N-acetyl-D-serine-p-fluorobenzylamide;
N-acetyl-D-phenylglycinebenzylamide;
D-1,2-(N,O-dimethylhydroxylamino)-2-acetamido acetic acid benzylamide; and
D-1,2-(O-methylhydroxylamino)-2-acetamido acetic acid benzylamide.

Depending upon the substituents, certain of the present compounds may form salts. For example, compounds of Formulas (I), (II) and (III) can form salts with a wide variety of acids, inorganic and organic, including pharmaceutically acceptable acids. Such salts can have enhanced water solubility and may be particularly useful in preparing pharmaceutical compositions for use in situations where enhanced water solubility is advantageous.

Pharmaceutically acceptable salts are those having therapeutic efficacy without unacceptable toxicity. Salts of inorganic acids such as hydrochloric, hydroiodic, hydrobromic, phosphoric, metaphosphoric, nitric and sulfuric acids as well as salts of organic acids such as tartaric, acetic, citric, malic, benzoic, perchloric, glycolic, gluconic, succinic, arylsulfonic (e.g., p-toluene sulfonic, benzenesulfonic), phosphoric and malonic acids and the like, can be used.

Compounds useful herein can be prepared by any known procedure of synthesis, for example as described in above-referenced U.S. Pat. No. 5,378,729 and U.S. Pat. No. 5,773,475, each of which is incorporated herein by reference.

A compound as described herein is used in a therapeutically effective amount. A physician can determine a suitable dosage of a compound, which can vary with the particular compound chosen, the route and method of administration, and the age and other characteristics of the individual patient. The physician can initiate treatment with small doses, for example substantially less than an optimum dose of the compound, and increase the dose by small increments until an optimum effect under the circumstances is achieved. When the composition is administered orally, larger quantities of the compound may be required to produce the same therapeutic benefit as a smaller quantity given parenterally.

In a particular aspect, the compound, for example lacosamide, is administered in an amount ranging from about 1 mg to about 10 mg per kilogram of body weight per day. Typically a patient can be treated with the compound, for example lacosamide, at a dose of at least about 50 mg/day, for example at least about 100 mg/day, at least about 200 mg/day, at least about 300 mg/day or at least about 400 mg/day. Generally, a suitable dose is not greater than about 6 g/day, for example not greater than about 1 g/day or not greater than about 600 mg/day. In some cases, however, higher or lower doses may be needed.

In another aspect, the daily dose is increased until a predetermined daily dose is reached which is maintained during further treatment.

In yet another aspect, several divided doses are administered daily. For example, no more than three doses per day, or no more than two doses per day, may be administered. However, it is often most convenient to administer no more than a single dose per day.

Doses expressed herein on a daily basis, for example in mg/day, are not to be interpreted as requiring a once-a-day frequency of administration. For example, a dose of 300 mg/day can be given as 100 mg three times a day, or as 600 mg every second day.

In yet another aspect, an amount of the compound, for example lacosamide, is administered which results in a plasma concentration of the compound of about 0.1 to about 15 μg/ml (trough) and about 5 to about 18.5 μg/ml (peak), calculated as an average over a plurality of treated subjects.

The compound of Formulas (I), (II) or (III), for example lacosamide, can be administered in any convenient and effective manner, such as by oral, intravenous, intraperitoneal, intramuscular, intrathecal, subcutaneous or transmucosal (e.g., buccal) routes. Oral or intravenous administration is generally preferred.

For oral administration, the compound is typically administered as a component of an orally deliverable pharmaceutical composition that further comprises an inert diluent or an assimilable edible carrier, or it may be incorporated into the subject's food. In an orally deliverable pharmaceutical composition, the compound can be incorporated together with one or more excipients and administered in the form of tablets, troches, pills, capsules, elixirs, suspensions, syrups, wafers, or the like. Such compositions typically contain at least about 1%, more typically about 5% to about 80%, by weight of the compound, for example lacosamide. The amount of the compound in the composition is such that, upon administration of the composition, a suitable dosage as set forth above can conveniently be provided. Illustratively, a pharmaceutical composition useful for oral delivery of a compound of Formulas (I), (II) or (III), for example lacosamide, contains about 10 mg to about 6 g, for example about 50 to about 1000 mg, or about 100 to about 600 mg, of the compound.

In particular embodiments the composition is enclosed in hard or soft shell (e.g., gelatin) capsules, or is in a form of compressed or molded tablets. The composition illustratively comprises as excipients one or more of a diluent such as lactose or dicalcium phosphate (in the case of capsules a liquid carrier can be present); a binding agent such as gum tragacanth, acacia, corn starch or gelatin; a disintegrating agent such as corn starch, potato starch, alginic acid or the like; a lubricant such as magnesium stearate; and a sweetening agent such as sucrose or saccharin and/or a flavoring agent such as peppermint, oil of wintergreen, or cherry flavoring can be added if desired.

Various other excipients may be present as coatings or otherwise modifying the physical form of the composition. For example, tablets, pills, or capsules may be coated with shellac, sugar or both. A syrup or elixir may contain the active compound, sucrose as a sweetening agent, methyl- and propylparabens as preservatives, a dye, and flavoring such as cherry or orange flavor. The active compound can be incorporated into a sustained-release formulation. For example, sustained-release dosage forms are contemplated wherein the compound is bound to an ion exchange resin which, optionally, can be coated with a diffusion barrier coating to modify the release properties of the resin.

Pharmaceutical compositions suitable for injectable use include sterile aqueous solutions (where the compound is water soluble), dispersions, and sterile powders for extemporaneous preparation of sterile injectable solutions or dispersions. In such cases the injectable composition must be sterile and must be sufficiently fluid to permit easy syringeability. The composition must be stable under the conditions of manufacture and storage and must typically be preserved against the contaminating action of microorganisms such as bacteria and fungi. The carrier can be a solvent or dispersion medium containing, for example, water, ethanol, polyol (for example, glycerol, propylene glycol, liquid polyethylene glycol, or the like), suitable mixtures thereof, and vegetable oils. Proper fluidity can be maintained, for example, by use of a coating such as lecithin, by maintenance of a required particle size in the case of dispersions, and by use of surfactants. Microbial action can be inhibited by various antibacterial and antifungal agents, for example, parabens, chlorobutanol, phenol, sorbic acid, thimerosal, or the like. In many cases, it will be preferable to include tonicity agents, for example, sugars or sodium chloride, to provide a substantially isotonic liquid for injection. Prolonged absorption of injectable compositions can be brought about by use in the compositions of agents delaying absorption, for example aluminum monostearate or gelatin.

Sterile injectable solutions can be prepared by incorporating the active compound in a required amount in an appropriate solvent with various of the other ingredients mentioned above, as required, followed by filtered sterilization. Generally, dispersions are prepared by incorporating sterilized active compound into a sterile vehicle which contains the dispersion medium and other excipient ingredients such as those mentioned above. Sterile powders for preparation of sterile injectable solutions can be prepared by vacuum-drying or freeze-drying a previously sterile-filtered solution or dispersion.

Administration of the compound, for example lacosamide, according to the present method is useful for treating non-inflammatory osteoarthritic pain. In particular, such administration is useful when non-inflammatory osteoarthritic pain is associated with trauma, joint damage, joint damage by trauma, cartilage degradation, structural bone changes, and/or vascularization of areas of bone remodeling. Bone continually undergoes remodeling. Remodeling is a process in which old bone is replaced with new bone to maintain peak bone density. Vascularization occurs by the proliferation of capillaries during the remodeling process and can be increased in conditions such as osteoarthritis.

In one embodiment, the non-inflammatory osteoarthritic pain is characterized by the absence of swelling or warmth, absence of inflammatory and/or systemic features, and/or essentially no morning stiffness.

Since cartilage is aneural, osteoarthritic pain can be mediated by effects of cartilage degradation on surrounding structures, such as bone and joint capsule. As used herein, cartilage degradation includes but is not limited to cartilage erosion, loss of surface integrity, cleft formation, and increased erosion with joint motion.

In the present invention, structural bone changes include, but are not limited to subchrondral bone thickening, microfractures, osteonecrosis, osteonecrosis beneath the bone surface, and weakened bone support of the cartilage.

In the present invention, osteoarthritic pain includes, but is not limited to osteoarthritic pain in the knee, hip, hand, and/or spine.

In one embodiment, administration of the compound, for example lacosamide, according to the present method inhibits transmission of pain. Inhibition of transmission of pain may be accomplished by preventing spinal neurons from responding to neurotransmitters, such as glutamate, released by nociceptors.

In some embodiments, the method of the present invention comprises administering a compound of Formulas (I), (II) or (III), for example lacosamide, in combination with a second active agent effective for treating, preventing or alleviating non-inflammatory osteoarthritic pain.

The two or more active agents of such a combination can be formulated in one pharmaceutical preparation (single dosage form) for administration to the subject at the same time, or in two or more distinct preparations (separate dosage forms) for administration to the subject at the same or different times, e.g., sequentially. The two distinct preparations can be formulated for administration by the same route or by different routes.

The second active agent may comprise a compound different from that of Formulas (I), (II) or (III), and may in particular comprise an anticonvulsant, for example selected from first generation anticonvulsants, such as carbamazepine and phenyloin, and second generation anticonvulsants, such as gabapentin, pregabalin, lamotrigine and levetiracetam.

More generally, the second agent can comprise one or more anticonvulsants selected from acetylpheneturide, albutoin, aminoglutethimide, 4-amino-3-hydroxybutyric acid, atrolactamide, beclamide, buramate, carbamazepine, cinromide, clomethiazole, clonazepam, decimemide, diethadione, dimethadione, doxenitoin, eterobarb, ethadione, ethosuximide, ethotoin, felbamate, fluoresone, fosphenyloin, gabapentin, ganaxolone, lamotrigine, levetiracetam, lorazepam, mephenyloin, mephobarbital, metharbital, methetoin, methsuximide, midazolam, narcobarbital, nitrazepam, oxcarbazepine, paramethadione, phenacemide, phenetharbital, pheneturide, phenobarbital, phensuximide, phenylmethylbarbituric acid, phenyloin, phenethylate, pregabalin, primidone, progabide, remacemide, rufinamide, suclofenide, sulthiame, talampanel, tetrantoin, tiagabine, topiramate, trimethadione, valproic acid, valpromide, vigabatrin, zonisamide, pharmaceutically acceptable salts thereof, and combinations thereof. In an illustrative example, the second agent comprises gabapentin.

In yet another aspect, the method comprises administering, in combination or adjunctive therapy with the compound of Formulas (I), (II) or (III), for example lacosamide, at least one anti-osteoarthritis agent other than an anticonvulsant.

The at least one anti-osteoarthritis agent can be effective for treatment (including prevention) of osteoarthritis or any aspect, symptom or underlying cause thereof. In one embodiment, the anti-osteoarthritis agent is effective for treatment of pain, i.e., analgesia. Suitable analgesics include opioid and non-opioid analgesics as well as certain anti-inflammatory drugs (see immediately below).

As indicated herein, osteoarthritis pain can comprise both a non-inflammatory and inflammatory component. Therefore, in another embodiment the anti-osteoarthritis agent is effective for treating inflammation and/or pain related thereto. Suitable anti-inflammatories include steroidal and nonsteroidal anti-inflammatory drugs. Nonsteroidal anti-inflammatory drugs (NSAIDs) include traditional NSAIDs and cyclooxygenase-2 (COX-2) selective inhibitors.

Nonlimiting examples of opioid and non-opioid analgesics that can be useful as the anti-osteoarthritis agent for administration in combination or adjunctive therapy with a compound of Formulas (I), (II) or (III), e.g., lacosamide, include acetaminophen, alfentanil, allylprodine, alphaprodine, anileridine, benzylmorphine, bezitramide, buprenorphine, butorphanol, clonitazene, codeine, cyclazocine, desomorphine, dextromoramide, dextropropoxyphene, dezocine, diampromide, diamorphone, dihydrocodeine, dihydromorphine, dimenoxadol, dimepheptanol, dimethylthiambutene, dioxaphetyl butyrate, dipipanone, dipyrone (metamizol), eptazocine, ethoheptazine, ethylmethylthiambutene, ethylmorphine, etonitazene, fentanyl, heroin, hydrocodone, hydromorphone, hydroxypethidine, isomethadone, ketobemidone, levallorphan, levorphanol, levophenacyl-morphan, lofentanil, meperidine, meptazinol, metazocine, methadone, metopon, morphine, myrophine, nalbuphine, nalorphine, narceine, nicomorphine, norlevorphanol, normethadone, normorphine, norpipanone, opium, oxycodone, oxymorphone, papaveretum, pentazocine, phenadoxone, phenazocine, phenomorphan, phenoperidine, piminodine, piritramide, proheptazine, promedol, properidine, propiram, propoxyphene, sufentanil, tilidine, tramadol, NO-naproxen, NCX-701, ALGRX-4975, pharmaceutically acceptable salts thereof, and combinations thereof. In an illustrative example, the anti-osteoarthritis agent comprises morphine or a pharmaceutically acceptable salt thereof.

Nonlimiting examples of steroidal anti-inflammatories that can be useful as the anti-osteoarthritis agent for administration in combination or adjunctive therapy with a compound of Formulas (I), (II) or (III), e.g., lacosamide, include alclometasone, amcinonide, betamethasone, betamethasone 17-valerate, clobetasol, clobetasol propionate, clocortolone, cortisone, dehydrotestosterone, deoxycorticosterone, desonide, desoximetasone, dexamethasone, dexamethasone 21-isonicotinate, diflorasone, fluocinonide, fluocinolone, fluorometholone, flurandrenolide, fluticasone, halcinonide, halobetasol, hydrocortisone, hydrocortisone acetate, hydrocortisone cypionate, hydrocortisone hemisuccinate, hydrocortisone 21-lysinate, hydrocortisone sodium succinate, isoflupredone, isoflupredone acetate, methylprednisolone, methylprednisolone acetate, methylprednisolone sodium succinate, methylprednisolone suleptanate, mometasone, prednicarbate, prednisolone, prednisolone acetate, prednisolone hemisuccinate, prednisolone sodium phosphate, prednisolone sodium succinate, prednisolone valerate-acetate, prednisone, triamcinolone, triamcinolone acetonide, pharmaceutically acceptable salts thereof, and combinations thereof.

Nonlimiting examples of NSAIDs that can be useful as the anti-osteoarthritis agent for administration in combination or adjunctive therapy with a compound of Formulas (I), (II) or (III), e.g., lacosamide, include salicylic acid derivatives (such as salicylic acid, acetylsalicylic acid, methyl salicylate, diflunisal, olsalazine, salsalate and sulfasalazine), indole and indene acetic acids (such as indomethacin, etodolac and sulindac), fenamates (such as etofenamic, meclofenamic, mefenamic, flufenamic, niflumic and tolfenamic acids), heteroaryl acetic acids (such as acemetacin, alclofenac, clidanac, diclofenac, fenchlofenac, fentiazac, furofenac, ibufenac, isoxepac, ketorolac, oxipinac, tiopinac, tolmetin, zidometacin and zomepirac), aryl acetic acid and propionic acid derivatives (such as alminoprofen, benoxaprofen, bucloxic acid, carprofen, fenbufen, fenoprofen, fluprofen, flurbiprofen, ibuprofen, indoprofen, ketoprofen, miroprofen, naproxen, naproxen sodium, oxaprozin, pirprofen, pranoprofen, suprofen, tiaprofenic acid and tioxaprofen), enolic acids (such as the oxicam derivatives ampiroxicam, cinnoxicam, droxicam, lomoxicam, meloxicam, piroxicam, sudoxicam and tenoxicam, and the pyrazolone derivatives aminopyrine, antipyrine, apazone, dipyrone, oxyphenbutazone and phenylbutazone), alkanones (such as nabumetone), nimesulide, proquazone, MX-1094, licofelone, pharmaceutically acceptable salts thereof, and combinations thereof.

Nonlimiting examples of COX-2 selective inhibitors that can be useful as the anti-osteoarthritis agent for administration in combination or adjunctive therapy with a compound of Formulas (I), (II) or (III), e.g., lacosamide, include celecoxib, deracoxib, valdecoxib, parecoxib, rofecoxib, etoricoxib, lumiracoxib, 2-(3,5-difluorophenyl)-3-[4-(methylsulfonyl)phenyl]-2-cyclopenten-1-one, (S)-6,8-dichloro-2-(trifluoromethyl)-2H-1-benzopyran-3-carboxylic acid, 2-(3,4-difluorophenyl)-4-(3-hydroxy-3-methyl-1-butoxy)-5-[4-(methylsulfonyl)phenyl]-3-(2H)-pyridazinone, 4-[5-(4-fluorophenyl)-3-(trifluoromethyl)-1H-pyrazol-1-yl]benzenesulfonamide, 4-[5-(phenyl)-3-(trifluoromethyl)-1H-pyrazol-1-yl]benzenesulfonamide, PAC-10549, cimicoxib, GW-406381, LAS-34475, CS-502, pharmaceutically acceptable salts thereof, and combinations thereof.

In yet another embodiment, the anti-osteoarthritis agent is a disease-modifying osteoarthritis drug (DMOAD). The term “DMOAD” herein refers to any drug that has utility in treatment of osteoarthritis or symptoms thereof, other than those mentioned above. Nonlimiting examples of DMOADs that can be useful as the anti-osteoarthritis agent for administration in combination or adjunctive therapy with a compound of Formulas (I), (II) or (III), e.g., lacosamide, include methotrexate, diacerein, glucosamine, chondroitin sulfate, anakinra, MMP inhibitors, doxycycline, minocycline, misoprostol, proton pump inhibitors, non-acetylated salicylates, tamoxifen, prednisone, methylprednisolone, polysulfated glycosaminoglycan, calcitonin, alendronate, risedronate, zoledronic acid, teriparatide, VX-765, pralnacasan, SB-462795, CPA-926, ONO-4817, S-3536, PG-530742, CP-544439, pharmaceutically acceptable salts thereof, and combinations thereof.

In yet another embodiment, the anti-osteoarthritis agent is a symptom-modifying osteoarthritis drug other than those mentioned above. Nonlimiting examples of such drugs that can be useful as the anti-osteoarthritis agent for administration in combination or adjunctive therapy with a compound of Formulas (I), (II) or (III), e.g., lacosamide, include ADL-100116, AD-827, HOE-140, DA-5018, pharmaceutically acceptable salts thereof, and combinations thereof.

In yet another aspect, the method comprises administering, in combination or adjunctive therapy with the compound of Formulas (I), (II) or (III), for example lacosamide, at least one antidepressant. Such combination or adjunctive therapies can, in some situations, be more effective in treatment of non-inflammatory osteoarthritic pain and/or have reduced adverse side effects than monotherapies with the compound of Formulas (I), (II) or (III), for example lacosamide, or the antidepressant alone.

Nonlimiting examples of antidepressants that can be useful in combination or adjunctive therapy with a compound of Formulas (I), (II) or (III), e.g., lacosamide, include without limitation bicyclic, tricyclic and tetracyclic antidepressants, hydrazides, hydrazines, phenyloxazolidinones and pyrrolidones. Specific examples include adinazolam, adrafinil, amineptine, amitriptyline, amitriptylinoxide, amoxapine, befloxatone, bupropion, butacetin, butriptyline, caroxazone, citalopram, clomipramine, cotinine, demexiptiline, desipramine, dibenzepin, dimetacrine, dimethazan, dioxadrol, dothiepin, doxepin, duloxetine, etoperidone, femoxetine, fencamine, fenpentadiol, fluacizine, fluoxetine, fluvoxamine, hematoporphyrin, hypericin, imipramine, imipramine N-oxide, indalpine, indeloxazine, iprindole, iproclozide, iproniazid, isocarboxazid, levophacetoperane, lofepramine, maprotiline, medifoxamine, melitracen, metapramine, metralindole, mianserin, milnacipran, minaprine, mirtazapine, moclobemide, nefazodone, nefopam, nialamide, nomifensine, nortriptyline, noxiptilin, octamoxin, opipramol, oxaflozane, oxitriptan, oxypertine, paroxetine, phenelzine, piberaline, pizotyline, prolintane, propizepine, protriptyline, pyrisuccideanol, quinupramine, reboxetine, ritanserin, roxindole, rubidium chloride, sertraline, sulpiride, tandospirone, thiazesim, thozalinone, tianeptine, tofenacin, toloxatone, tranylcypromine, trazodone, trimipramine, tryptophan, venlafaxine, viloxazine, zimeldine, pharmaceutically acceptable salts thereof, and combinations thereof.

In yet another aspect, the method comprises administering, in combination or adjunctive therapy with the compound of Formulas (I), (II) or (III), for example lacosamide, at least one NMDA receptor antagonist. Such combination or adjunctive therapies can, in some situations, be more effective in treatment of non-inflammatory osteoarthritic pain and/or have reduced adverse side effects than monotherapies with the compound of Formulas (I), (II) or (III), for example lacosamide, or the NMDA receptor antagonist alone.

Nonlimiting examples of NMDA receptor antagonists that can be useful in combination or adjunctive therapy with a compound of Formulas (I), (II) or (III), e.g., lacosamide, include amantadine, D-AP5, aptiganel, CPP, dexanabinol, dextromethorphan, dextropropoxyphene, 5,7-dichlorokynurenic acid, gavestinel, ifendopril, ketamine, ketobemidone, licostinel, LY-235959, memantine, methadone, MK-801, phencyclidine, remacemide, selfotel, tiletamine, pharmaceutically acceptable salts thereof, and combinations thereof.

Suitable regimens including doses and routes of administration for particular anti-osteoarthritis agents can be determined from readily-available reference sources relating to these agents, for example Physicians' Desk Reference (PDR), 60th edition, Montvale, NJ: Thomson (2006) and various internet sources known to those of skill in the art. When administered in combination or adjunctive therapy with a compound of Formulas (I), (II) or (III), for example lacosamide, the anti-osteoarthritis agent can be used at a full dose, but the physician may elect to administer less than a full dose of the anti-osteoarthritis agent, at least initially.

More than one anti-osteoarthritis agent can be administered in combination or adjunctive therapy with a compound of Formulas (I), (II) or (III), for example lacosamide. In one embodiment two or more such agents are included in the combination or adjunctive therapy, selected from two or more of the following classes:

(i) opioid and non-opioid analgesics;

(ii) steroidal anti-inflammatories;

(iii) NSAIDs and COX-2 selective inhibitors; and

(iv) DMOADs.

The compound of Formulas (I), (II) or (III), for example lacosamide, and the anti-osteoarthritis agent can be administered together, i.e., in a single coformulated dosage form, or separately, i.e., as components of two separate dosage forms. Separate dosage forms can be administered substantially at the same time or at different times or frequencies.

The term “therapeutic combination” refers to a plurality of agents that, when administered to a subject together or separately, are co-active in bringing therapeutic benefit to the subject. Such administration is referred to as “combination therapy,” “co-therapy,” “adjunctive therapy” or “add-on therapy.” For example, one agent can potentiate or enhance the therapeutic effect of another, or reduce an adverse side effect of another, or one or more agents can be effectively administered at a lower dose than when used alone, or can provide greater therapeutic benefit than when used alone, or can complementarily address different aspects, symptoms or etiological factors of a disease or condition.

In an embodiment of the present invention, a therapeutic combination is provided comprising a compound of Formulas (I), (II) or (III), for example lacosamide, and a second active agent effective for treating non-inflammatory osteoarthritic pain, such as, for example, an anticonvulsant. Suitable anticonvulsants are listed above.

In another embodiment, a therapeutic combination is provided comprising a compound of Formulas (I), (II) or (III), for example lacosamide, and at least one anti-osteoarthritis agent other than an anticonvulsant. Suitable anti-osteoarthritic agents are listed above.

In yet another embodiment, a therapeutic combination is provided comprising a compound of Formulas (I), (II) or (III), for example lacosamide, and at least one antidepressant. Suitable antidepressants are listed above.

In yet another embodiment, a therapeutic combination is provided comprising a compound of Formulas (I), (II) or (III), for example lacosamide, and at least one NMDA receptor antagonist. Suitable NMDA receptor antagonists are listed above.

The two or more active agents of the above combinations can be formulated in one pharmaceutical preparation (single dosage form) for administration to the subject at the same time, or in two or more distinct preparations (separate dosage forms) for administration to the subject at the same or different times, e.g., sequentially. The two distinct preparations can be formulated for administration by the same route or by different routes.

Separate dosage forms can optionally be co-packaged, for example in a single container or in a plurality of containers within a single outer package, or co-presented in separate packaging (“common presentation”). As an example of co-packaging or common presentation, a kit is contemplated comprising, in a first container, the compound of Formulas (I), (II) or (III) and, in a second container, the second active agent or the at least one anti-osteoarthritis agent. In another example, the compound of Formulas (I), (II) or (III) and the second active agent or the at least one anti-osteoarthritis agent are separately packaged and available for sale independently of one another, but are co-marketed or co-promoted for use according to the invention. The separate dosage forms may also be presented to a subject separately and independently, for use according to the invention.

Depending on the dosage forms, which may be identical or different, e.g., fast release dosage forms, controlled release dosage forms or depot forms, the compound of Formulas (I), (II) or (III) and the second active agent or the anti-osteoarthritis agent may be administered on the same or on different schedules, for example on a daily, weekly or monthly basis.

### EXAMPLES

### Example 1

This example describes a study demonstrating antinociceptive effectiveness of lacosamide in inhibiting mechanical hyperalgesia, as measured by paw withdrawal threshold to muscle pressure, and mechanical allodynia, as measured by biceps muscle grip strength, occurring in musculoskeletal pain induced by TNF in rats. The model used in this example is applicable to musculoskeletal pain which occurs in fibromyalgia, myofascial pain syndrome, back pain or osteoarthritis. For comparative purposes, the non-opioid analgesic dipyrone (metamizol) and the anticonvulsants pregabalin and gabapentin were included in the study.

Animals, Induction of Muscle Pain

Adult male Sprague Dawley rats with a body weight of 250 g to 300 g were used (supplier: Charles River, Sulzfeld, Germany). Animals were group-housed (3 animals per cage) and maintained in a room with controlled temperature (21-22° C.) and a reversed light-dark cycle (12 h/12 h) with food and water available ad libitum. All experiments were approved by the Bavarian State animal experimentation committee and carried out in accordance with its regulations.

Recombinant rat tumor necrosis factor alpha (herein referred to as TNF) was obtained from R&D Systems, Minneapolis, Minn., U.S.A. TNF was diluted in 0.9% NaCl and used in a concentration of 1 μg in 50 μl. Injections were performed in short halothane narcosis with a 30 g needle bilaterally into the gastrocnemius or into the biceps brachii muscle. All rats were used to the behavioral tests before injections and baseline values were recorded over three test days.

Behavioral Readout: Muscle Pressure (Randall-Selitto)

Mechanical withdrawal thresholds to muscle pressure were measured with an analgesimeter (Ugo Basile, Comerio, Italy). The rat was allowed to crawl into a sock which helps the rat to relax. The hind limbs were positioned such that an increasing pressure could be applied onto the gastrocnemius muscle (maximum 250 g). The pressure needed to elicit withdrawal was recorded. Means of 3 trials for each hind limb were calculated (interstimulus interval of >30 sec). Only animals with a significant TNF effect were included for further analysis.

Rats were injected with TNF into the gastrocnemius muscle at 2 pm. Eighteen hours later, rats were tested for pressure hyperalgesia pre- and post-administration of the test drug. Rats were tested for pressure hyperalgesia 30 to 60 minutes after drug administration.

Behavioral Readout: Grip Strength

Grip strength of the forelimbs was tested with a digital grip force meter (DFIS series, Chatillon, Greensboro, NC, U.S.A.). The rat was positioned to grab the grid with the forelimbs and was gently pulled so that the grip strength could be recorded. Means of three trials were calculated. The effect of the TNF treatment was calculated for each animal separately and only animals with a significant TNF effect were included for further analysis.

Rats were injected with TNF into the biceps brachii muscle at 8 am. Six hours later, grip strength of the forelimbs was tested with a digital grip force meter. Test drug was administered, and grip strength was again tested after 30 to 60 minutes.

Administration Protocol

The rats, initially 10 per group, were treated with either 3, 10 or 30 mg/kg lacosamide, 2 mg/kg metamizol, 30 or 100 mg/kg pregabalin, 100 mg/kg gabapentin, or the NaCl vehicle, i.p. (intraperitoneally). Volume of i.p. injections was 0.5 ml. A pilot study was performed to confirm that i.m. (intramuscular) injection of 1 μg TNF into the gastrocnemius muscle was sufficient to induce pressure hyperalgesia.

Groups and treatments are summarized for gastrocnemius muscle and biceps brachii muscle injections of TNF respectively in Tables 1 and 2.

TABLE 1
Gastrocnemius muscle injection of TNF

| Group no. | Induction treatment | Drug and dose | No. of rats |
|---|---|---|---|
| 1.1 | TNF 1 μg i.m. | lacosamide 3 mg/kg i.p. | 8 |
| 1.2 | TNF 1 μg i.m. | lacosamide 10 mg/kg i.p. | 8 |
| 1.3 | TNF 1 μg i.m. | lacosamide 30 mg/kg i.p. | 7 |
| 1.4 | TNF 1 μg i.m. | pregabalin 30 mg/kg i.p. | 8 |
| 1.5 | TNF 1 μg i.m. | pregabalin 100 mg/kg i.p. | 10 |
| 1.6 | TNF 1 μg i.m. | gabapentin 100 mg/kg i.p. | 10 |
| 1.7 | TNF 1 μg i.m. | NaCl vehicle i.p. | 10 |
| 1.8 | TNF 1 μg i.m. | metamizol 2 mg/kg i.p. | 9 |

TABLE 2
Biceps brachii injection of TNF

| Group no. | Induction treatment | Drug and dose | No. of rats |
|---|---|---|---|
| 2.1 | TNF 1 μg i.m. | lacosamide 3 mg/kg i.p. | 4 |
| 2.2 | TNF 1 μg i.m. | lacosamide 10 mg/kg i.p. | 9 |
| 2.3 | TNF 1 μg i.m. | lacosamide 30 mg/kg i.p. | 10 |
| 2.4 | TNF 1 μg i.m. | pregabalin 30 mg/kg i.p. | 10 |
| 2.5 | TNF 1 μg i.m. | pregabalin 100 mg/kg i.p. | 10 |
| 2.6 | TNF 1 μg i.m. | gabapentin 100 mg/kg i.p. | 10 |
| 2.7 | TNF 1 μg i.m. | NaCl vehicle i.p. | 10 |
| 2.8 | TNF 1 μg i.m. | metamizol 2 mg/kg i.p. | 7 |

Data Presentation and Statistics

Data are shown in graphs displaying means and standard errors of the means (SEM). Pre- and post-treatment data were compared using ANOVA (analysis of variance) and a Tukey post hoc test. Means of treatment groups were compared using a one-way ANOVA and Dunnett's post hoc test. Maximal possible effects (MPE) were calculated for all types of treatment.

Results: Muscle Pressure Hyperalgesia

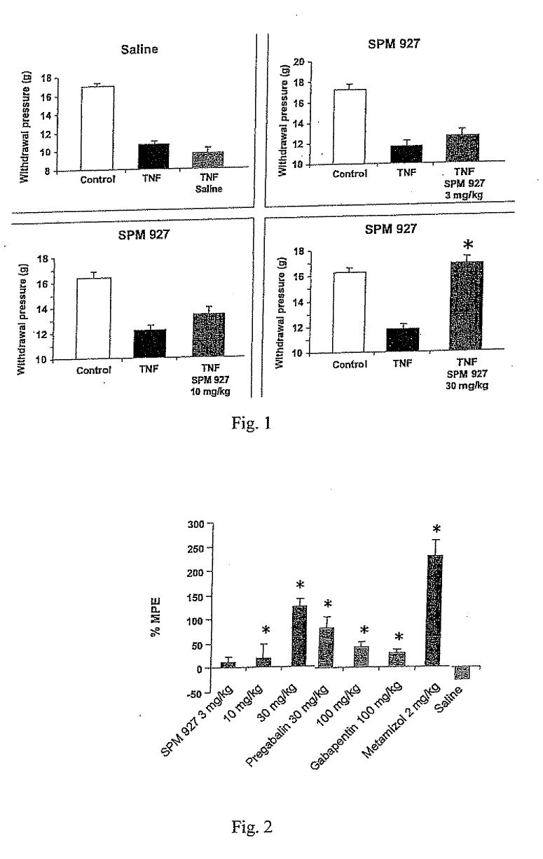

Only rats in which withdrawal thresholds were significantly reduced after TNF injection were included. In about 13% of the rats, the TNF effect was absent. FIG. 1 shows absolute values of withdrawal thresholds to pressure.

A complete reversal of muscular mechanical hyperalgesia was seen with lacosamide at 30 mg/kg and metamizol at 2 mg/kg.

A significant reversal of muscular mechanical hyperalgesia was also seen for pregabalin at 30 and 100 mg/kg, and gabapentin at 100 mg/kg.

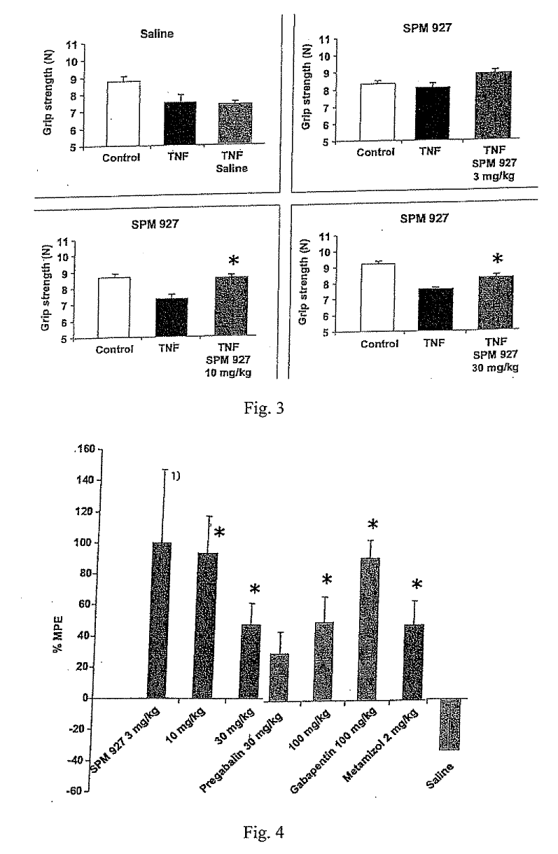

The MPE (FIG. 2) was significantly different from vehicle for lacosamide at 10 and 30 mg/kg, for pregabalin at 30 and 100 mg/kg, for gabapentin at 100 mg/kg, and for metamizol at 2 mg/kg. The vehicle had no effect.

Results: Biceps Muscle Grip Strength

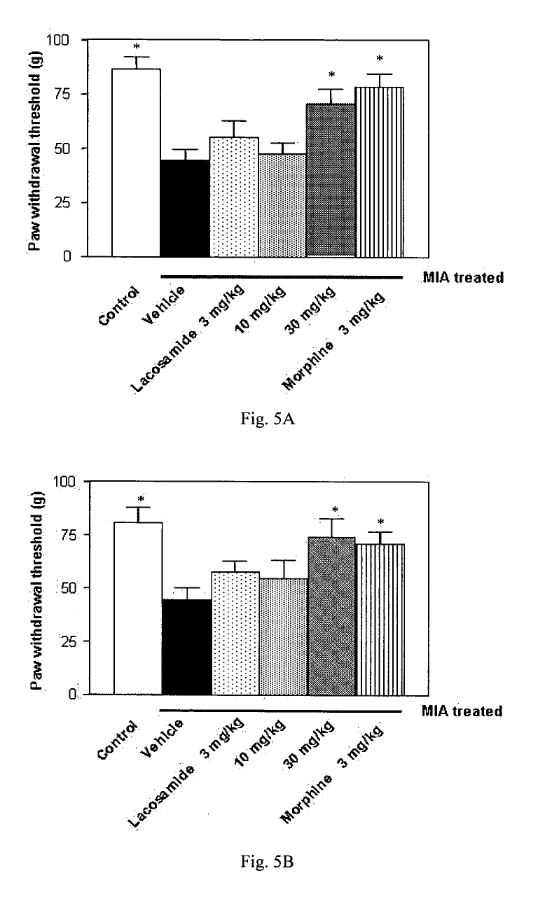

Only rats in which grip strength was significantly reduced after TNF injection were included. In about 13% of the rats, the TNF effect was absent. FIG. 3 shows absolute values of grip strength.

A significant reversal of TNF-induced reduction of grip strength was seen with lacosamide at 10 and 30 mg/kg. A significant reversal was also seen for pregabalin at 100 mg/kg, gabapentin at 100 mg/kg and metamizol at 2 mg/kg.

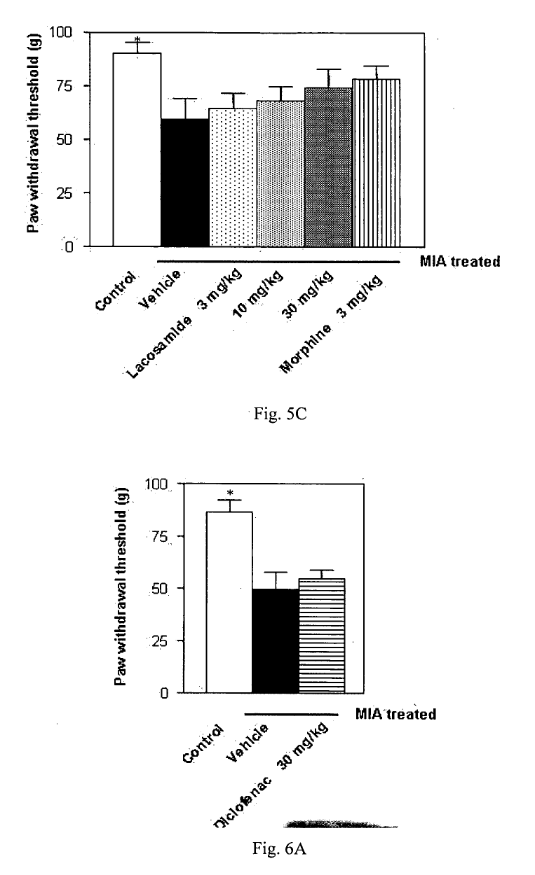

The MPE (FIG. 4) was significantly different from vehicle for lacosamide at 10 and 30 mg/kg, for pregabalin at 100 mg/kg, for gabapentin at 100 mg/kg, and for metamizol at 2 mg/kg. The vehicle had no effect.

Discussion

Lacosamide dose-dependently improved muscle hyperalgesia induced by TNF injection in the paw pressure test, reaching full reversal at 30 mg/kg. In comparison to the anticonvulsants pregabalin and gabapentin, lacosamide had a stronger effect on muscle pain. Neither pregabalin nor gabapentin led to a full reversal of the muscle hyperalgesia. In the grip strength test indicative of mechanical allodynia, lacosamide reversed the effect of TNF on the muscle at 10 mg/kg. Again lacosamide was more potent than pregabalin and gabapentin, which improved grip strength at 100 mg/kg.

In conclusion, lacosamide was effective in reducing the muscular hyperalgesia and mechanical allodynia induced by TNF injected into muscle. Thus lacosamide, illustratively of compounds of Formulas (I), (II) and (III), is concluded to have therapeutic efficacy in the treatment, in particular systemic treatment, of specific manifestations of non-inflammatory musculoskeletal pain, such as muscular hyperalgesia and allodynia, occurring for example in fibromyalgia, myofascial pain syndrome, back pain or osteoarthritis.

### Example 2

This example describes a study demonstrating antinociceptive effectiveness of lacosamide in an iodoacetate rat model. The model used in this example is applicable to non-inflammatory osteoarthritic pain. For comparative purposes, the opioid analgesic morphine and the NSAID diclofenac was included in the study.

One of the best characterized rat models for osteoarthritis is injection of the metabolic inhibitor monosodium iodoacetate into a joint, for example a knee joint, which inhibits activity of glyceraldehyde-3-phosphate dehydrogenase in chondrocytes, resulting in disruption of glycolysis and eventually in cell death (Guzman et al. (2003) Toxicol. Pathol. 31(6):619-624; Kalbhen (1987) J. Rheumatol. 14(Spec. No.):130-131). The progressive loss of chondrocytes results in histological and morphological changes of the articular cartilage, closely resembling those seen in human osteoarthritis patients.

Animals

Male Wistar rats (Janview, France) weighing 170-200 g at the start of the study were used. The animals were group-housed (3 animals per cage) in a room with controlled temperature (21-22° C.), and a reversed light-dark cycle (12 h/12 h), and had free access to food and water.

Induction of Osteoarthritis

Osteoarthritis was induced by intra-articular injection in 50 μl of 3 mg monosodium iodoacetate (MIA) (Sigma) through the intrapatellar ligament of the right knee. Control rats were injected with an equivalent volume of saline. Up to five days after the iodoacetate injection a substantial inflammation of synovial joints was observed in this model. The general health of the animals was monitored. No signs of distress were seen.

Histology

On each of days 3, 7 and 14 after iodoacetate treatment, four animals were sacrificed for histology study. Knees were harvested and fixed overnight in 10% formalin and subsequently decalcified with 10% formic acid for 72 h before being embedded in paraffin. Sections 10 μm thick were prepared every 250 μm. Hematoxylin/eosin staining was carried out to assess the extent of inflammatory infiltrates in the joints and surrounding tissues, and Saflanin-O fast green staining was done to measure the degeneration of cartilage.

Evaluation of the Effect of Compounds on Nociception

In the first round of experiments the iodoacetate-treated rats were randomized to six experimental groups (12 animals per group) which received the following treatments (p.o.=per os; s.c.=subcutaneous) on the days of pain assessment (days 3, 7 and 14 post-iodoacetate treatment):

p.o. injection of saline (vehicle);

p.o. injection of 3 mg/kg lacosamide;

p.o. injection of 10 mg/kg lacosamide;

p.o. injection of 30 mg/kg lacosamide;

s.c. injection of 3 mg/kg morphine.

Diclofenac (30 mg/kg, s.c.) was tested in a separate experiment by the same scientists under the same conditions at about the same time. The non-iodoacetate treated control group (control) received p.o. injection of saline 45 minutes prior to the pain assessment. Lacosamide, diclofenac and morphine were injected 60 minutes prior to implementation of behavioral tests. Each group was examined blind.

Evaluation of Tactile Allodynia and Mechanical Hyperalgesia

For testing tactile allodynia, rats were placed on a metallic grid floor. Nociceptive testing was done by inserting a von Frey filament (Bioseb, France) through the grid floor and applying it to the plantar surface of the hind paw. A trial consisted of several applications of different von Frey filaments (at a frequency of about 1 Hz). The von Frey filaments were applied from filament 10 g to 100 g. As soon as the animal removed its hind paw, the test was stopped and the filament number was recorded to represent the paw withdrawal threshold.

For testing mechanical hyperalgesia, nociceptive flexion reflexes were quantified using the Randall-Selitto paw pressure device (Bioseb, France), which applied a linearly increasing mechanical force to the dorsum of the rat's hind paw. The paw withdrawal threshold was defined as the force at which the rat withdrew its paw. The cutoff pressure was set to 250 g.

Drugs and Reagents

Lacosamide (Schwarz BioSciences GmbH) and morphine sulfate (Francopia, France) were dissolved in saline. Monosodium iodoacetate and diclofenac were purchased from Sigma (France). Drug administration was made in a volume of 1 ml/kg.

Data Analyses and Statistics

Comparisons of groups of behavioral data at each individual time point were conducted using ANOVA followed by post-hoc analysis (Dunnett's test).

Results

Joint pathology was assessed on day 3, 7 and 14 following intra-articular injection of iodoacetate. At day 3 there was a substantial initial inflammatory response. This inflammation was characterized by an expansion of the synovial membrane most likely caused by proteinaceous edema fluid and fibrin with infiltrating macrophages, neutrophils, plasma cells and lymphocytes. The cartilage was still intact. By day 7, inflammation within the synovium and surrounding tissue has largely resolved. On day 14 proteoglycan loss was seen throughout the depth of the cartilage. The synovial membrane looked normal and contained no inflammatory cells.

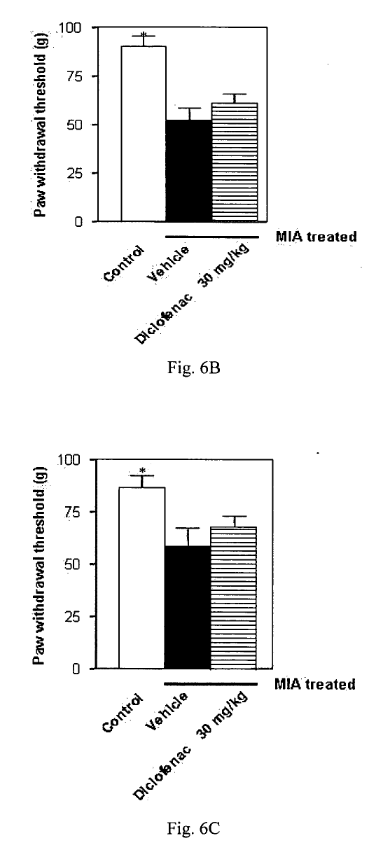

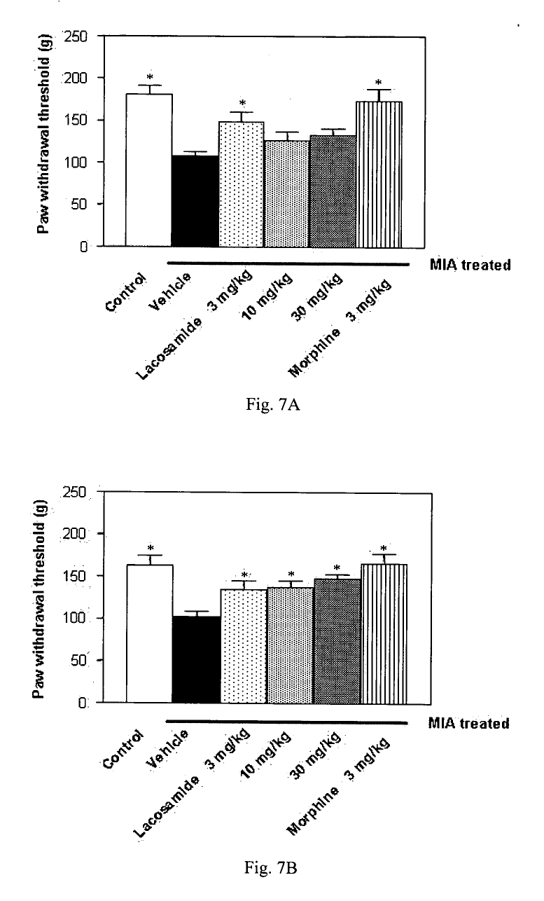

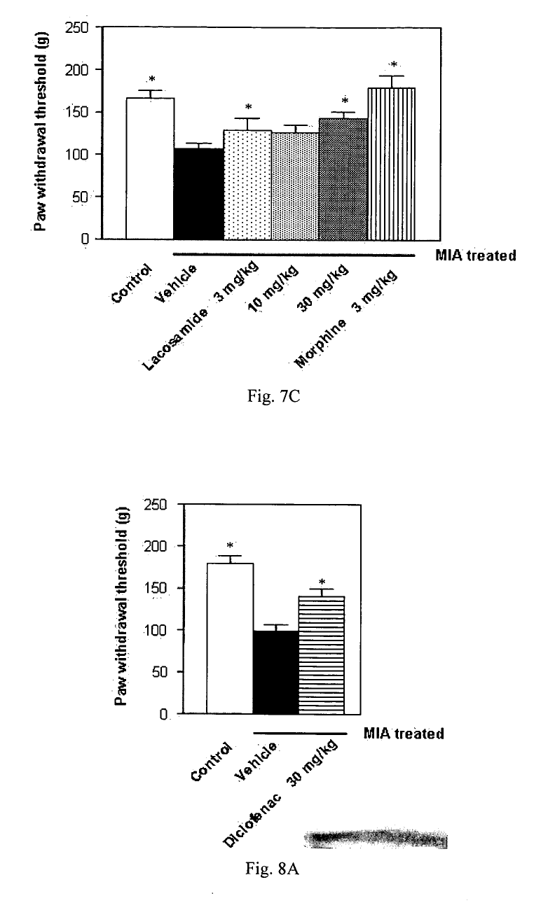

Tactile allodynia, tested with von Frey filaments, was assessed at day 3, 7, and 14 in iodoacetate-treated rats compared to control rats. Treatment with lacosamide (30 mg/kg) and morphine (3 mg/kg) improved tactile allodynia of iodoacetate-treated rats at day 3 (FIG. 5A) and 7 (FIG. 5B) but not on day 14 (FIG. 5C), and lower doses of lacosamide showed a trend for such improvement. Diclofenac (30 mg/kg) had no effect on tactile allodynia at day 3 (FIG. 6A), day 7 (FIG. 6B) or day 14 (FIG. 6C).

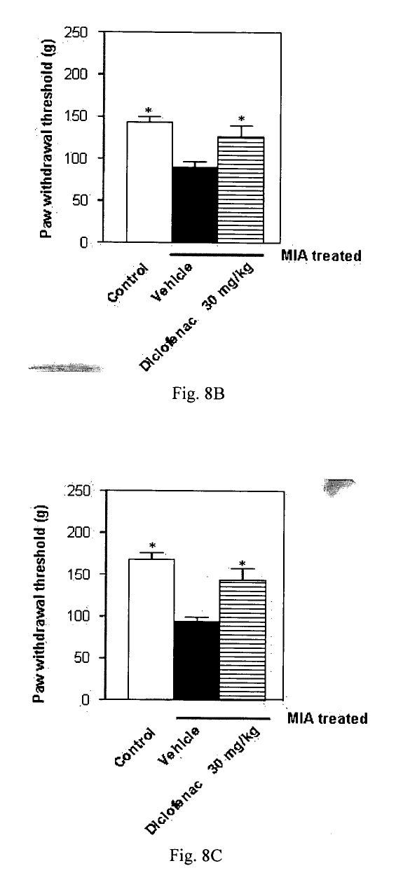

There was a marked mechanical hyperalgesia as evidenced by a reduction in the paw pressure withdrawal thresholds in the iodoacetate/vehicle treated animals compared to control/vehicle treated animals. Treatment of iodoacetate-treated rats with lacosamide 3 mg/kg, morphine 3 mg/kg and diclofenac 30 mg/kg induced in each case an increase in paw pressure withdrawal threshold compared to iodoacetate/vehicle treated animals on day 3 (FIGS. 7A, 8A). On day 7, lacosamide at all doses tested (3, 10 and 30 mg/kg), morphine and diclofenac each reduced mechanical hyperalgesia (FIGS. 7B, 8B). Similar results were seen at day 14 after iodoacetate treatment except that the group treated with 10 mg/kg lacosamide did not show a statistically significant effect (FIGS. 7C, 8C). Interestingly, in the iodoacetate-treated animals, mechanical hyperalgesia developed from day 3 and lasted for at least 14 days, compared to tactile allodynia which was more pronounced during the early phase of arthritis development, reflecting an ongoing development of pain sensitization based on different molecular mechanisms during the 14 days post iodoacetate-treatment.

The results show that lacosamide inhibited mechanical hyperalgesia during the post-inflammatory period, indicating effectiveness of lacosamide for treating non-inflammatory osteoarthritic pain.

### Example 3

This example describes a study demonstrating effectiveness of lacosamide alone and in combination with gabapentin in the rat formalin paw test (late phase), as described by Wheeler-Aceto & Cowan (1991) Psychopharmacology 104:35-44, which detects analgesic activity.

Materials and Methods

Rats were given an intraplantar injection of 5% formalin (50 μl) into the posterior left paw. This treatment induces a recognizable flinching and licking response of the affected paw in control animals. The number of flinches was counted for 15 minutes, beginning 20 minutes after injection of formalin. The time spent licking the affected paw was also recorded.

Male Rj: Wistar (Han) rats, 10 per group, weighing 100-130 g at the beginning of the experiments were studied per group. The test was performed blind.

Lacosamide (20 mg/kg), gabapentin (50 and 100 mg/kg), combinations of lacosamide (20 mg/kg) with gabapentin (50 and 100 mg/kg), and vehicle were administered i.p. 10 minutes before injection of formalin.

Results

Results of the test are presented in Tables 3 (number of flinches) and 4 (licking time).

TABLE 3
Effect of lacosamide, gabapentin and combinations on number of flinches

| Compound 1 | Compound 2 | No. of flinches | | |
|---|---|---|---|---|
| (mg/kg) | (mg/kg) | mean ± SEM | p value | % change |
| Vehicle | Vehicle | 127.8 ± 21.2 | — | — |
| Lacosamide | Vehicle | 85.7 ± 14.3 NS (a) | 0.1736 | −33% (a) |
| (20) | | | | |
| Vehicle | Gabapentin | 97.4 ± 23.8 NS (a) | 0.3445 | −24% (a) |
| | (50) | | | |
| Vehicle | Gabapentin | 88.1 ± 19.4 NS (a) | 0.2121 | −31% (a) |
| | (100) | | | |
| Lacosamide | Gabapentin | ** (a) | 0.0071 | −64% (a) |
| (20) | (50)# | 46.0 ± 21.1 * (b) | 0.0222 | −46% (b) |
| | | NS (c) | 0.0790 | −53% (c) |
| Lacosamide | Gabapentin | ** (a) | 0.0017 | −76% (a) |
| (20) | (100) | 31.0 ± 9.3 ** (b) | 0.0041 | −64% (b) |
| | | * (c) | 0.0343 | −65% (c) |

TABLE 4
Effect of lacosamide, gabapentin and combinations on licking time

| Compound 1 | Compound 2 | Licking time (seconds) | | |
|---|---|---|---|---|
| (mg/kg) | (mg/kg) | mean ± SEM | p value | % change |
| Vehicle | Vehicle | 222.4 ± 33.8 | — | — |
| Lacosamide | Vehicle | 146.9 ± 23.8 NS (a) | 0.0962 | −34% (a) |
| (20) | | | | |
| Vehicle | Gabapentin | 161.0 ± 27.3 NS (a) | 0.2258 | −28% (a) |
| | (50) | | | |
| Vehicle | Gabapentin | 90.0 ± 22.5 * (a) | 0.0101 | −60% (a) |
| | (100) | | | |
| Lacosamide | Gabapentin | ** (a) | 0.0042 | −74% (a) |
| (20) | (50)# | 58.6 ± 32.0 * (b) | 0.0220 | −60% (b) |
| | | * (c) | 0.0365 | −64% (c) |
| Lacosamide | Gabapentin | *** (a) | 0.0007 | −82% (a) |
| (20) | (100) | 39.1 ± 19.9 ** (b) | 0.0022 | −73% (b) |
| | | NS (c) | 0.0685 | −57% (c) |

NS = not significant;
* = p < 0.05;
** = p < 0.01;
*** = p < 0.001
(a): compared with vehicle control
(b): compared with lacosamide alone at the appropriate dose
(c): compared with gabapentin alone at the appropriate dose
#missing value ( 1/10)

Lacosamide alone at 20 mg/kg tended to decrease the number of flinches by 33% as compared with vehicle controls. It also tended to decrease the time spent licking, by 34% as compared with vehicle controls (p=0.0962).

Gabapentin alone at 50 and 100 mg/kg globally but non-significantly decreased the number of flinches, by 24% and 31% respectively as compared with vehicle controls. Gabapentin dose-dependently decreased the time spent licking, by 28% (50 mg/kg) and 60% (100 mg/kg), significantly so at 100 mg/kg (p<0.05).

Lacosamide 20 mg/kg combined with gabapentin 50 and 100 mg/kg clearly and dose-dependently decreased the number of flinches, by 64% and 76% respectively (p<0.01) as compared with vehicle controls. The combination clearly and dose-dependently decreased the time spent licking, by 74% (p<0.01) and 82% (p<0.001) respectively. The effects of lacosamide combined with gabapentin on the number of flinches and the time spent licking were significantly more marked than the effects of lacosamide alone (p <0.05 or p<0.01).

### Example 4

This example describes a study demonstrating effectiveness of lacosamide alone and in combination with morphine in the rat formalin paw test (late phase), as described by Wheeler-Aceto & Cowan (1991), supra.

Materials and Methods

Test methods were similar to those of Example 3. Lacosamide (10 and 20 mg/kg), morphine (2 and 4 mg/kg), combinations of lacosamide (10 and 20 mg/kg) with morphine (2 and 4 mg/kg), and vehicle were administered i.p. 10 minutes before injection of formalin.

Results

Results of the test are presented in Tables 5 (number of flinches) and 6 (licking time).

TABLE 5
Effect of lacosamide, morphine and combinations on number of flinches

| Compound 1 | Compound 2 | No. of flinches | | |
|---|---|---|---|---|
| (mg/kg) | (mg/kg) | mean ± SEM | p value | % change |
| Vehicle | Vehicle | 150.0 ± 21.0 | — | — |
| Lacosamide | Vehicle | 182.7 ± 25.9 NS (a) | 0.3254 | +22% (a) |
| (10) | | | | |
| Lacosamide | Vehicle | 97.2 ± 16.0 NS (a) | 0.0961 | −35% (a) |
| (20) | | | | |
| Vehicle | Morphine (2) | 139.5 ± 25.3 NS (a) | 0.6499 | −7% (a) |
| Vehicle | Morphine (4) | 94.3 ± 21.1 NS (a) | 0.1303 | −37% (a) |
| Lacosamide | Morphine (2) | NS (a) | 0.7621 | −7% (a) |
| (10) | | 139.7 ± 29.4 NS (b) | 0.3638 | −24% (b) |
| | | NS (c) | 0.8205 | 0% (c) |
| Lacosamide | Morphine (4) | *** (a) | 0.0002 | −86% (a) |
| (10) | | 20.6 ± 7.9 *** (b) | 0.0003 | −89% (b) |
| | | ** (c) | 0.0035 | −78% (c) |
| Lacosamide | Morphine (2) | ** (a) | 0.0015 | −70% (a) |
| (20) | | 44.7 ± 12.3 * (b) | 0.0342 | −54% (b) |
| | | ** (c) | 0.0091 | −68% (c) |
| Lacosamide | Morphine (4) | ***(a) | 0.0005 | −87% (a) |
| (20) | | 19.6 ± 13.3 ** (b) | 0.0014 | −80% (b) |
| | | ** (c) | 0.0024 | −79% (c) |

TABLE 6
Effect of lacosamide, morphine and combinations on licking time

| Compound 1 | Compound 2 | Licking time (seconds) | | |
|---|---|---|---|---|
| (mg/kg) | (mg/kg) | mean ± SEM | p value | % change |
| Vehicle | Vehicle | 291.9 ± 25.6 | — | — |
| Lacosamide | Vehicle | 210.1 ± 22.7 * (a) | 0.0191 | −28% (a) |
| (10) | | | | |
| Lacosamide | Vehicle | 128.2 ± 28.0 *** (a) | 0.0009 | −56% (a) |
| (20) | | | | |
| Vehicle | Morphine (2) | 289.3 ± 30.7 NS (a) | 0.7054 | −1% (a) |
| Vehicle | Morphine (4) | 234.9 ± 37.3 NS (a) | 0.4055 | −20% (a) |
| Lacosamide | Morphine (2) | NS (a) | 0.1304 | −27% (a) |
| (10) | | 19.6 ± 13.3 NS (b) | 0.7624 | +1% (b) |
| | | * (c) | 0.0284 | −27% (c) |
| Lacosamide | Morphine (4) | ** (a) | 0.0051 | −48% (a) |
| (10) | | 150.9 ± 36.3 NS (b) | 0.2265 | −28% (b) |
| | | NS (c) | 0.1306 | −36% (c) |
| Lacosamide | Morphine (2) | *** (a) | 0.0004 | −69% (a) |
| (20) | | 91.5 ± 25.7 NS (b) | 0.2258 | −29% (b) |
| | | *** (c) | 0.0009 | −68% (c) |
| Lacosamide | Morphine (4) | *** (a) | 0.0001 | −94% (a) |
| (20) | | 17.1 ± 16.4 ** (b) | 0.0018 | −87% (b) |
| | | *** (c) | 0.0003 | −93% (c) |

NS = not significant;
* = p < 0.05;
** = p < 0.01;
*** = p < 0.001
(a): compared with vehicle control
(b): compared with lacosamide alone at the appropriate dose
(c): compared with morphine alone at the appropriate dose

Lacosamide alone at 10 and 20 mg/kg did not strongly affect the number of flinches, as compared with vehicle controls (22% increase and 35% decrease, respectively) although the tendency towards a decrease at 20 mg/kg approached statistical significance (p=0.0961). Lacosamide dose-dependently decreased the time spent licking by 28% (p<0.05) at 10 mg/kg and by 56% (p<0.001) at 20 mg/kg.

Morphine alone at 2 and 4 mg/kg dose-dependently decreased the number of flinches and the time spent licking, as compared with vehicle controls. Nevertheless, these effects did not reach statistical significance.

Lacosamide 10 mg/kg combined with morphine 4 mg/kg, but not with morphine 2 mg/kg, clearly decreased the number of flinches by 86% (p<0.001) and the time spent licking by 48% (p<0.01), as compared with vehicle controls. The effects of lacosamide 10 mg/kg combined with morphine 4 mg/kg on the number of flinches, but not on the time spent licking, were more marked than the effects of lacosamide alone at the same dose (p<0.001).

Lacosamide 20 mg/kg combined with morphine 2 and 4 mg/kg clearly and dose-dependently decreased the number of flinches by 70% (p<0.01) and 87% (p<0.001) respectively, as compared with vehicle controls. The combination clearly and dose-dependently decreased the time spent licking by 69% and 94%, respectively (p<0.001). The effects of lacosamide 20 mg/kg combined with morphine on the number of flinches and the time spent licking were significantly more marked than the effects of lacosamide alone at the same dose (p<0.05 or p<0.01), except for the time spent licking at the 2 mg/kg dose of morphine.

### Example 5

This example describes a study demonstrating effectiveness of lacosamide alone and in combination with the antidepressant duloxetine in the rat formalin paw test (late phase), as described by Wheeler-Aceto & Cowan (1991), supra.

Materials and Methods

Test methods were similar to those of Example 3. Lacosamide (10 mg/kg), duloxetine (8 mg/kg), a combination of lacosamide (10 mg/kg) with duloxetine (8 mg/kg), and vehicle were administered i.p. 10 minutes before injection of formalin.

Results

Results of the test are presented in Tables 7 (number of flinches) and 8 (licking time).

TABLE 7
Effect of lacosamide, duloxetine and combination on number of flinches

| Compound 1 | Compound 2 | No. of flinches | | |
|---|---|---|---|---|
| (mg/kg) | (mg/kg) | mean ± SEM | p value | % change |
| Vehicle | Vehicle | 151.3 ± 13.7 | — | — |
| Lacosamide | Vehicle | 158.2 ± 15.6 NS (a) | 0.5963 | +5% (a) |
| (10) | | | | |
| Vehicle | Duloxetine (8) | 149.6 ± 27.3 NS (a) | 0.7054 | −1% (a) |
| Lacosamide | Duloxetine (8) | * (a) | 0.0233 | −31% (a) |
| (10) | | 105.1 ± 11.3 * (b) | 0.0284 | −34% (b) |
| | | NS (c) | 0.1988 | −30% (c) |

TABLE 8
Effect of lacosamide, duloxetine and combination on licking time

| Compound 1 | Compound 2 | Licking time (seconds) | | |
|---|---|---|---|---|
| (mg/kg) | (mg/kg) | mean ± SEM | p value | % change |
| Vehicle | Vehicle | 264.2 ± 17.8 | — | — |
| Lacosamide | Vehicle | 185.2 ± 31.7 NS (a) | 0.0538 | −30% (a) |
| (10) | | | | |
| Vehicle | Duloxetine (8) | 195.5 ± 45.0 NS (a) | 0.1615 | −26% (a) |
| Lacosamide | Duloxetine (8) | *** (a) | 0.0004 | −63% (a) |
| (10) | | 96.9 ± 24.8 * (b) | 0.0340 | −48% (b) |
| | | NS (c) | 0.1492 | −50% (c) |

NS = not significant;
* = p < 0.05;
** = p < 0.01;
*** = p < 0.001
(a): compared with vehicle control
(b): compared with lacosamide alone at the appropriate dose
(c): compared with duloxetine alone at the appropriate dose

Lacosamide 10 mg/kg alone had no significant effects although it tended to decrease the time spent licking (30% decrease, p=0.0538).

Duloxetine 8 mg/kg alone had no clear effects.

Lacosamide 10 mg/kg combined with duloxetine 8 mg/kg significantly decreased the number of flinches, as compared with vehicle controls, by 31% (p<0.05). The combination decreased the time spent licking by 63% (p<0.001). The effects of lacosamide combined with duloxetine on the number of flinches and the time spent licking were more marked than the effects of lacosamide alone (p<0.05 to p<0.01).

### Example 6

This example describes a study demonstrating effectiveness of lacosamide alone and in combination with the NMDA receptor antagonist memantine in the rat formalin paw test (late phase), as described by Wheeler-Aceto & Cowan (1991), supra.

Materials and Methods

Test methods were similar to those of Example 3. Lacosamide (10 and 20 mg/kg), memantine (4 and 8 mg/kg), combinations of lacosamide (10 and 20 mg/kg) with memantine (4 and 8 mg/kg), and vehicle were administered i.p. 10 minutes before injection of formalin.

Results

Results of the test are presented in Tables 9 (number of flinches) and 10 (licking time).

TABLE 9
Effect of lacosamide, memantine and combinations on number of flinches

| Compound 1 | Compound 2 | No. of flinches | | |
|---|---|---|---|---|
| (mg/kg) | (mg/kg) | mean ± SEM | p value | % change |
| Vehicle | Vehicle | 165.6 ± 20.1 | — | — |
| Lacosamide | Vehicle | 113.9 ± 23.2 NS (a) | 0.0821 | −31% (a) |
| (10) | | | | |
| Lacosamide | Vehicle | 85.8 ± 14.4 * (a) | 0.0101 | −48% (a) |
| (20) | | | | |
| Vehicle | Memantine (4) | 161.4 ± 26.3 NS (a) | 0.7052 | −3% (a) |
| Vehicle | Memantine (8) | 132.3 ± 24.6 NS (a) | 0.3845 | −20% (a) |
| Lacosamide | Memantine (4) | * (a) | 0.0211 | −36% (a) |
| (10) | | 105.4 ± 16.1 NS (b) | 0.8205 | −7% (b) |
| | | NS (c) | 0.1124 | −35% (c) |
| Lacosamide | Memantine (8) | * (a) | 0.0311 | −50% (a) |
| (10) | | 83.5 ± 23.4 NS (b) | 0.2568 | −27% (b) |
| | | NS (c) | 0.1988 | −37% (c) |
| Lacosamide | Memantine (4) | *** (a) | 0.0004 | −74% (a) |
| (20) | | 42.5 ± 9.0 * (b) | 0.0257 | −50% (b) |
| | | *** (c) | 0.0004 | −74% (c) |
| Lacosamide | Memantine (8) | *** (a) | 0.0007 | −64% (a) |
| (20) | | 59.6 ± 11.0 NS (b) | 0.1986 | −31% (b) |
| | | * (c) | 0.0283 | −55% (c) |

TABLE 10
Effect of lacosamide, memantine and combinations on licking time

| Compound 1 | Compound 2 | Licking time (seconds) | | |
|---|---|---|---|---|
| (mg/kg) | (mg/kg) | mean ± SEM | p value | % change |
| Vehicle | Vehicle | 176.3 ± 18.2 | — | — |
| Lacosamide (10) | Vehicle | 168.5 ± 23.9 NS (a) | 0.8797 | −4% (a) |
| Lacosamide (20) | Vehicle | 85.1 ± 19.1 ** (a) | 0.0072 | −52% (a) |
| Vehicle | Memantine (4) | 219.9 ± 21.8 NS (a) | 0.0537 | +25% (a) |
| Vehicle | Memantine (8) | 237.3 ± 18.9 * (a) | 0.0412 | +35% (a) |
| Lacosamide (10) | Memantine (4)# | NS (a) | 0.7749 | −5% (a) |
| | | 168.2 ± 26.1 NS (b) | 0.9349 | 0% (b) |
| | | NS (c) | 0.1208 | −24% (c) |
| Lacosamide (10) | Memantine (8) | * (a) | 0.0342 | −35% (a) |
| | | 114.8 ± 18.8 NS (b) | 0.1508 | −32% (b) |
| | | ** (c) | 0.0015 | −52% (c) |
| Lacosamide (20) | Memantine (4) | *** (a) | 0.0002 | −69% (a) |
| | | 54.1 ± 10.5 NS (b) | 0.3071 | −36% (b) |
| | | *** (c) | 0.0007 | −75% (c) |
| Lacosamide (20) | Memantine (8) | * (a) | 0.0191 | −49% (a) |
| | | 90.6 ± 26.8 NS (b) | 0.8500 | +6% (b) |
| | | ** (c) | 0.0015 | −62% (c) |

NS = not significant;
* = p < 0.05;
** = p < 0.01;
*** = p < 0.001
(a): compared with vehicle control
(b): compared with lacosamide alone at the appropriate dose
(c): compared with memantine alone at the appropriate dose
#missing value ( 1/10)

Lacosamide alone at 10 and 20 mg/kg dose-dependently decreased the number of flinches, as compared with vehicle controls, by 31% and 48% respectively, significantly so at 20 mg/kg (p<0.05). Lacosamide clearly decreased the time spent licking at 20 mg/kg (52% decrease, p<0.01) but had no clear effects at 10 mg/kg.

Memantine alone at 4 and 8 mg/kg did not clearly affect the number of flinches, as compared with vehicle controls. Memantine dose-dependently increased the time spent licking (25% increase, p=0.0537 and 35% increase, p<0.05).

Lacosamide at 10 mg/kg combined with memantine at 4 and 8 mg/kg dose-dependently decreased the number of flinches, as compared with vehicle controls, by 36% and 50% respectively (p<0.05). The combination significantly decreased the time spent licking at 8 but not at 4 mg/kg of memantine (35% decrease, p<0.05). The effects of lacosamide combined with memantine on the number of flinches and the time spent licking were not different from the effects of lacosamide alone.

Lacosamide at 20 mg/kg combined with memantine at 4 and 8 mg/kg clearly decreased the number of flinches, as compared with vehicle controls, by 74% and 64% respectively (p<0.001). The combination clearly decreased the time spent licking, although in a manner inversely related to the dose of memantine (69% decrease, p<0.001 and 49% decrease, p<0.05, respectively). The effects of lacosamide combined with memantine at 4 mg/kg on the number of flinches but not on the time spent licking were significantly more marked than the effects of lacosamide alone (p<0.05).

### Example 7

This example describes a study demonstrating effectiveness of lacosamide alone and in combination with naproxen in the rat formalin paw test (late phase), as described by Wheeler-Aceto & Cowan (1991), supra.

Materials and Methods

Test methods were similar to those of Example 3. Lacosamide (10 and 20 mg/kg), naproxen (8 and 16 mg/kg), combinations of lacosamide (10 and 20 mg/kg) with memantine (8 and 16 mg/kg), and vehicle were administered i.p. 10 minutes before injection of formalin. Morphine (8 mg/kg) was included as a comparative treatment.

Results

Results of the test are presented in Tables 11 (number of flinches) and 12 (licking time).

TABLE 11
Effect of lacosamide, naproxen and combinations on number of flinches

| Compound 1 | Compound 2 | No. of flinches | | |
|---|---|---|---|---|
| (mg/kg) | (mg/kg) | mean ± SEM | p value | % change |
| Vehicle | Vehicle | 114.1 ± 21.7 | — | — |
| Lacosamide (10) | Vehicle# | 99.2 ± 16.6 NS (a) | 0.6828 | −13% (a) |
| Lacosamide (20) | Vehicle | 100.3 ± 22.3 NS (a) | 0.6501 | −12% (a) |
| Vehicle | Naproxen (8) | 148.0 ± 35.5 NS (a) | 0.5453 | +30% (a) |
| Vehicle | Naproxen (16) | 116.6 ± 20.7 NS (a) | 0.9698 | +2% (a) |
| Lacosamide (10) | Naproxen (8) | 143.5 ± 33.1 NS (a) | 0.4494 | +26% (a) |
| | | NS (b) | 0.4624 | +45% (b) |
| | | NS (c) | 0.9698 | −3% (c) |
| Lacosamide (10) | Naproxen (16) | 103.7 ± 18.6 NS (a) | 0.7336 | −9% (a) |
| | | NS (b) | 0.9674 | +5% (b) |
| | | NS (c) | 0.7336 | −11% (c) |
| Lacosamide (20) | Naproxen (8) | 104.2 ± 18.4 NS (a) | 0.7623 | −9% (a) |
| | | NS (b) | 0.9397 | +4% (b) |
| | | NS (c) | 0.5202 | −30% (c) |
| Lacosamide (20) | Naproxen (16) | 77.7 ± 20.2 NS (a) | 0.1403 | −32% (a) |
| | | NS (b) | 0.3258 | −23% (b) |
| | | NS (c) | 0.1306 | −33% (c) |
| Morphine (8) | Vehicle | 0.2 ± 0.1 *** (a) | <0.0001 | −100% (a) |

TABLE 12
Effect of lacosamide, naproxen and combinations on licking time

| Compound 1 | Compound 2 | Licking time (seconds) | | |
|---|---|---|---|---|
| (mg/kg) | (mg/kg) | mean ± SEM | p value | % change |
| Vehicle | Vehicle | 191.1 ± 11.5 | — | — |
| Lacosamide (10) | Vehicle# | 174.7 ± 20.4 NS (a) | 0.5401 | −9% (a) |
| Lacosamide (20) | Vehicle | 78.9 ± 20.4 ***(a) | 0.0007 | −59% (a) |
| Vehicle | Naproxen (8) | 222.1 ± 21.1 NS (a) | 0.3258 | +16% (a) |
| Vehicle | Naproxen (16) | 190.1 ± 25.4 NS (a) | 0.6775 | −1% (a) |
| Lacosamide (10) | Naproxen (8) | 178.3 ± 31.6 NS (a) | 0.7336 | −7% (a) |
| | | NS (b) | 0.6242 | +2% (b) |
| | | NS (c) | 0.4963 | −20% (c) |
| Lacosamide (10) | Naproxen (16) | 118.3 ± 24.9 * (a) | 0.0211 | −38% (a) |
| | | NS (b) | 0.1651 | −32% (b) |
| | | * (c) | 0.0492 | −38% (c) |
| Lacosamide (20) | Naproxen (8) | 150.0 ± 28.2 NS (a) | 0.5706 | −22% (a) |
| | | NS (b) | 0.0584 | +90% (b) |
| | | NS (c) | 0.0696 | −32% (c) |
| Lacosamide (20) | Naproxen (16) | 89.8 ± 22.8 ** (a) | 0.0052 | −53% (a) |
| | | NS (b) | 0.7620 | +14% (b) |
| | | * (c) | 0.0126 | −53% (c) |
| Morphine (8) | Vehicle | 0.0 ± 0.0 *** (a) | <0.0001 | −100% (a) |

NS = not significant;
* = p < 0.05;
** = p < 0.01;
*** = p < 0.001
(a): compared with vehicle control
(b): compared with lacosamide alone at the appropriate dose
(c): compared with naproxen alone at the appropriate dose
#missing value ( 1/10)

Lacosamide alone at 10 and 20 mg/kg did not clearly affect the number of flinches, as compared with vehicle controls. It clearly decreased the time spent licking at 20 mg/kg, by 59% (p<0.001), but had no clear effects at 10 mg/kg.

Naproxen alone at 8 and 16 mg/kg did not clearly affect the number of flinches or the time spent licking, as compared with vehicle controls.

Lacosamide 10 mg/kg combined with naproxen 8 and 16 mg/kg did not clearly affect the number of flinches, as compared with vehicle controls. Lacosamide 10 mg/kg combined with naproxen at 16 but not at 8 mg/kg significantly decreased the time spent licking, by 38% (p<0.05). The effects of lacosamide 10 mg/kg combined with naproxen on the number of flinches and the time spent licking were not different from the effects of lacosamide alone.

Lacosamide 20 mg/kg combined with naproxen 8 and 16 mg/kg did not clearly affect the number of flinches, as compared with vehicle controls. Lacosamide 20 mg/kg combined with naproxen at 16 but not 8 mg/kg significantly decreased the time spent licking, by 53% (p<0.01). The effects of lacosamide 20 mg/kg combined with naproxen on the number of flinches and the time spent licking were not different from the effects of lacosamide alone.

Morphine alone at 8 mg/kg, administered under the same experimental conditions, eliminated flinching and the time spent licking, as compared with vehicle controls (p<0.001).

All patents and publications cited herein are incorporated by reference into this application in their entirety.

The words “comprise”, “comprises”, and “comprising” are to be interpreted inclusively rather than exclusively.

What is claimed is:

1. A method for treating non-inflammatory osteoarthritic pain in a subject, the method comprising administering to the subject a compound of Formula (I) 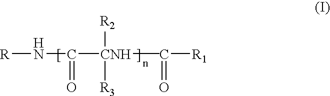
wherein:
R is hydrogen, lower alkyl, lower alkenyl, lower alkynyl, aryl, aryl lower alkyl, heterocyclic, heterocyclic lower alkyl, lower alkyl heterocyclic, lower cycloalkyl or lower cycloalkyl lower alkyl, and R is unsubstituted or is substituted with at least one electron withdrawing group, and/or at least one electron donating group;
R₁ is hydrogen or lower alkyl, lower alkenyl, lower alkynyl, aryl lower alkyl, aryl, heterocyclic lower alkyl, lower alkyl heterocyclic, heterocyclic, lower cycloalkyl, or lower cycloalkyl lower alkyl, and is unsubstituted or substituted with at least one electron-withdrawing group and/or at least one electron-donating group;
R₂ and R₃ are independently hydrogen, lower alkyl, lower alkenyl, lower alkynyl, aryl lower alkyl, aryl, halo, heterocyclic, heterocyclic lower alkyl, lower alkyl heterocyclic, lower cycloalkyl, lower cycloalkyl lower alkyl, or Z-Y, wherein R₂ and R₃ are each independently unsubstituted or substituted with at least one electron-withdrawing group and/or at least one electron-donating group;
Z is O, S, S(O)ₐ, NR₄, NR′₆, PR₄ or a chemical bond;
Y is hydrogen, lower alkyl, aryl, aryl lower alkyl, lower alkenyl, lower alkynyl, halo, heterocyclic, heterocyclic lower alkyl, or lower alkyl heterocyclic, and is unsubstituted or substituted with at least one electron-withdrawing group and/or at least one electron-donating group, provided that when Y is halo, Z is a chemical bond, or
Z-Y taken together is NR₄NR₅R₇, NR₄OR₅, ONR₄R₇, OPR₄R₅, PR₄OR₅, SNR₄R₇, NR₄SR₇, SPR₄R₅, PR₄SR₇, NR₄PR₅R₆, PR4NR5R7, N⁺R₅R₆R₇, 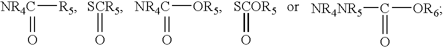
R′₆ is hydrogen, lower alkyl, lower alkenyl, or lower alkynyl, and is unsubstituted or substituted with at least one electron-withdrawing group or/and at least one electron-donating group;
R₄, R₅ and R₆ are independently hydrogen, lower alkyl, aryl, aryl lower alkyl, lower alkenyl, or lower alkynyl, and are each independently unsubstituted or substituted with at least one electron-withdrawing group or/and at least one electron-donating group;
R₇ is R₆, COOR₈, or COR₈, and is unsubstituted or substituted with at least one electron-withdrawing group or/and at least one electron-donating group;
R₈ is hydrogen, lower alkyl, or aryl lower alkyl, and is unsubstituted or substituted with at least one electron-withdrawing group or/and at least one electron-donating group;
n is 1-4; and
a is 1-3;
or a pharmaceutically acceptable salt thereof.

2. The method of claim 1, wherein, in the compound of Formula (I), one or both of R₂ and R₃ are heterocycles independently selected from the group consisting of furyl, thienyl, pyrazolyl, pyrrolyl, methylpyrrolyl, imidazolyl, indolyl, thiazolyl, oxazolyl, isothiazolyl, isoxazolyl, piperidyl, pyrrolinyl, piperazinyl, quinolyl, triazolyl, tetrazolyl, isoquinolyl, benzofuryl, benzothienyl, morpholinyl, benzoxazolyl, tetrahydrofuryl, pyranyl, indazolyl, purinyl, indolinyl, pyrazolindinyl, imidazolinyl, imidazolindinyl, pyrrolidinyl, furazanyl, N-methylindolyl, methylfuryl, pyridazinyl, pyrimidinyl, pyrazinyl, pyridyl, epoxy, aziridino, oxetanyl, azetidinyl, and when N is present in the heterocycle, N-oxides thereof; said heterocycles being independently unsubstituted or substituted with at least one electron-withdrawing group and/or at least one electron-donating group.

3. The method of claim 1, wherein the compound is of Formula (III) 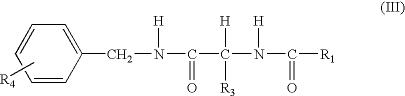
wherein:
R₄ is one or more substituents independently selected from the group consisting of hydrogen, halo, alkyl, alkenyl, alkynyl, nitro, carboxy, formyl, carboxyamido, aryl, quaternary ammonium, haloalkyl, aryl alkanoyl, hydroxy, alkoxy, amino, alkylamino, dialkylamino, aryloxy, mercapto, alkylthio, alkylmercapto and disulfide;
R₃ is selected from the group consisting of hydrogen, alkyl, alkoxy, alkoxyalkyl, aryl, N-alkoxy-N-alkylamino and N-alkoxyamino; and
R₁ is alkyl.

4. The method of claim 3, wherein, in the compound of Formula (III),
R₄ is one or more substituents independently selected from the group consisting of hydrogen and halo;
R₃ is selected from the group consisting of lower alkoxy lower alkyl, aryl, N-lower alkoxy-N-lower alkylamino, and N-lower alkoxyamino; and
R₁ is lower alkyl.

5. The method of claim 4, wherein, in the compound of Formula (III), R₃ is lower alkoxy lower alkyl.

6. The method of claim 3, wherein, in the compound of Formula (III), no more than one R₄ substituent is fluoro and all others are hydrogen;
R₃ is selected from the group consisting of methoxymethyl, phenyl, N-methoxy-N-methylamino, and N-methoxyamino; and
R₁ is methyl.

7. The method of claim 3, wherein, in the compound of Formula (III),
R₄ is hydrogen;
R₃ is methoxymethyl; and
R₁ is methyl.

8. The method of claim 3, wherein the compound of Formula (III) is selected from the group consisting of
(R)-2-acetamido-N-benzyl-3-methoxy-propionamide;
(R)-2-acetamido-N-benzyl-3-ethoxy-propionamide;
O-methyl-N-acetyl-D-serine-m-fluorobenzylamide;
O-methyl-N-acetyl-D-serine-p-fluorobenzylamide;
N-acetyl-D-phenylglycinebenzylamide;
D-1,2-(N,O-dimethylhydroxylamino)-2-acetamide acetic acid benzylamide; and
D-1,2-(O-methylhydroxylamino)-2-acetamide acetic acid benzylamide.

9. The method of claim 3, wherein the compound of Formula (III) is substantially enantiopure.

10. The method of claim 3, wherein the compound of Formula (III) is lacosamide.

11. The method of claim 10, wherein the lacosamide is administered at a dose of about 50 mg to about 6 g/day.

12. The method of claim 10, wherein the lacosamide is administered at a dose of about 100 to about 1000 mg/day.

13. The method of claim 10, wherein the lacosamide is administered at a dose of about 200 to about 600 mg/day.

14. The method of claim 10, wherein a peak plasma concentration of lacosamide of about 0.1 to about 15 μg/ml, calculated as an average over a plurality of treated subjects, is obtained.

15. The method of claim 3, wherein the compound of Formula (III) is administered according to a regimen wherein daily doses are increased until a predetermined daily dose is reached which is maintained during further treatment.

16. The method of claim 3, wherein the compound of Formula (III) is administered in one to three doses per day.

17. The method of claim 3, wherein the compound of Formula (III) is administered orally or intravenously.

18. The method of claim 1, wherein the osteoarthritic pain is associated with cartilage degradation, structural bone changes, and/or vascularization of areas of osteoarthritic bone remodeling.

19. The method of claim 1, further comprising administering a second active agent effective for treating non-inflammatory osteoarthritic pain.

20. The method of claim 19, wherein the second active agent is an anticonvulsant.

21. The method of claim 20, wherein the anticonvulsant is selected from the group consisting of carbamazepine, phenytoin, gabapentin, pregabalin, lamotrigine and levetiracetam.

22. The method of claim 1, further comprising administering at least one anti-osteoarthritis agent other than an anticonvulsant.

23. The method of claim 22, wherein the at least one anti-osteoarthritis agent is an opioid or non-opioid analgesic, a steroidal anti-inflammatory, an NSAID or COX-2 selective inhibitor, or a DMOAD.
